SEQUENCE LISTING (82 sequences)

SEQ ID NO: 1 - protein, 117 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNSLLDAMDYWGQGTSVTVSS

SEQ ID NO: 2 - protein, 10 aa, synthetic construct
GFSLTSYGVH

SEQ ID NO: 3 - protein, 16 aa, synthetic construct
VIWSGGSTDYNAAFIS

SEQ ID NO: 4 - protein, 9 aa, synthetic construct
NSLLDAMDY

SEQ ID NO: 5 - protein, 108 aa, synthetic construct
SIVMTQTPKFLLVSAGDRVTITCKASQSVSNAVAWYQQKPGQSPKLLIYYASNRYTGVPDRFTGSGYGTDFTFTISTVQAEDLAVYFCQQDYSSPLTFGAGTKLELKR

SEQ ID NO: 6 - protein, 11 aa, synthetic construct
KASQSVSNAVA

SEQ ID NO: 7 - protein, 7 aa, synthetic construct
YASNRYT

SEQ ID NO: 8 - protein, 9 aa, synthetic construct
QQDYSSPLT

SEQ ID NO: 9 - protein, 116 aa, synthetic construct
QVQLHQPGAELVKPGASVNLSCKASGYTFTSYWMHWVKQRPGQGLEWIGEINPSSGRANYNEKFKNKATLTVDKSSITAYMHLSSLTSEDSAVYYCHWDYYAMDYWGQGTSVTVSS

SEQ ID NO: 10 - protein, 10 aa, synthetic construct
GYTFTSYWMH

SEQ ID NO: 11 - protein, 17 aa, synthetic construct
EINPSSGRANYNEKFKN

SEQ ID NO: 12 - protein, 7 aa, synthetic construct
DYYAMDY

SEQ ID NO: 13 - protein, 108 aa, synthetic construct
DIVLTQSPATLSVTPGDSVSLSCRASQSISNNLHWYQQKSHESPRLLFKSASQSISGIPSRFSGSGSGTDFTLSINSVETEDFGMYFCQQTNSWPLTFGTGTKLDLKR

SEQ ID NO: 14 - protein, 11 aa, synthetic construct
RASQSISNNLH

SEQ ID NO: 15 - protein, 7 aa, synthetic construct
SASQSIS

SEQ ID NO: 16 - protein, 9 aa, synthetic construct
QQTNSWPLT

SEQ ID NO: 17 - protein, 116 aa, synthetic construct
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCATFYDGYDYWGQGTTLTVSS

SEQ ID NO: 18 - protein, 11 aa, synthetic construct
GYSITSGYYWN

SEQ ID NO: 19 - protein, 16 aa, synthetic construct
YISYDGSNNYNPSLKN

SEQ ID NO: 20 - protein, 7 aa, synthetic construct
FYDGYDY

SEQ ID NO: 21 - protein, 108 aa, synthetic construct
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPFTFGSGTKLEIKR

SEQ ID NO: 22 - protein, 11 aa, synthetic construct
KASQDVSTAVA

SEQ ID NO: 23 - protein, 7 aa, synthetic construct
WASTRHT

SEQ ID NO: 24 - protein, 9 aa, synthetic construct
QQHYSTPFT

SEQ ID NO: 25 - protein, 119 aa, synthetic construct
QVQLKESGPGLVAPSQSLSISCTVSGFSLTTFGIHWIRQPPGKGLEWLGVIWAAGTTNYNSTLKSRLTITKDNSRSQVFLKMNSLQTYDTAIYYCVREDYDYFFGLDYWGQGTSVTVSS

SEQ ID NO: 26 - protein, 10 aa, synthetic construct
GFSLTTFGIH

SEQ ID NO: 27 - protein, 16 aa, synthetic construct
VIWAAGTTNYNSTLKS

SEQ ID NO: 28 - protein, 11 aa, synthetic construct
EDYDYFFGLDY

SEQ ID NO: 29 - protein, 110 aa, synthetic construct
QAVVTQESALTTSPGETVTLTCRSSTGAVTTSNYASWVQEKPDHLFIGLIGGTNNRAPGVPARFSGSLIGDKAALTITGAQTDDEAIYFCALWFSNHWVFGGGTKLTVLG

SEQ ID NO: 30 - protein, 14 aa, synthetic construct
RSSTGAVTTSNYAS

SEQ ID NO: 31 - protein, 7 aa, synthetic construct
GTNNRAP

SEQ ID NO: 32 - protein, 9 aa, synthetic construct
ALWFSNHWV

SEQ ID NO: 33 - protein, 117 aa, synthetic construct
QVQLKESGPGLVAPSQSLSITCTVSGFSLTSYGVHWVRQPPGKGLEWLGVIWADGSTNYNSALMSRLSISKDNSKSQVFLKMNSLQTDDTAMYYCARWTYGDYFDYWGQGTTLTVSS

SEQ ID NO: 34 - protein, 16 aa, synthetic construct
VIWADGSTNYNSALMS

SEQ ID NO: 35 - protein, 9 aa, synthetic construct
WTYGDYFDY

SEQ ID NO: 36 - protein, 108 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 37 - protein, 11 aa, synthetic construct
SASQGISNYLN

SEQ ID NO: 38 - protein, 7 aa, synthetic construct
YTSSLHS

SEQ ID NO: 39 - protein, 9 aa, synthetic construct
QQYSKLPYT

SEQ ID NO: 40 - protein, 121 aa, synthetic construct
DVQLVESGGGLVQPGGSRKLSCAASGFTFSYFGMHWVRQAPEKGLEWVAYISSGSNTIYYADTVKGRFTISRDNPKNTLFLQMTSLRSEDTAMYYCARAYYGNHYYAMDFWGPGTSVTVSS

SEQ ID NO: 41 - protein, 10 aa, synthetic construct
GFTFSYFGMH

SEQ ID NO: 42 - protein, 17 aa, synthetic construct
YISSGSNTIYYADTVKG

SEQ ID NO: 43 - protein, 12 aa, synthetic construct
AYYGNHYYAMDF

SEQ ID NO: 44 - protein, 108 aa, synthetic construct
DIQMTQSPASLSVSVGETVTITCRASENIYSNLAWYQQKRGKSPQLLVHAARDLATGVPSRFSGSGSGTQYSLKINSLQSEDFGTYYCQHFWETPWTFGGGTKLEIKR

SEQ ID NO: 45 - protein, 11 aa, synthetic construct
RASENIYSNLA

SEQ ID NO: 46 - protein, 7 aa, synthetic construct
AARDLAT

SEQ ID NO: 47 - protein, 9 aa, synthetic construct
QHFWETPWT

SEQ ID NO: 48 - protein, 116 aa, synthetic construct
EVQLVESGGGLVKPGGSLKLSCAASGFTFSDYYMYWIRQTPEKRLEWVATISDGGFSTYYPDSVKGRFTISRDNAKNNLFLQMSSLKSEDTAIYYCARFGAYSTFWGQGTLVTVSS

SEQ ID NO: 49 - protein, 10 aa, synthetic construct
GFTFSDYYMY

SEQ ID NO: 50 - protein, 17 aa, synthetic construct
TISDGGFSTYYPDSVKG

SEQ ID NO: 51 - protein, 7 aa, synthetic construct
FGAYSTF

SEQ ID NO: 52 - protein, 108 aa, synthetic construct
QIVLSQSPAILSASPGEKVTMTCRASSSVSYMYWYQQKPGSSPKPWIYATSNLASGVPARFSGSGSGTSYSLTISRVEAEDAATYYCQQWSSDPPMTFGGGTKLEIKR

SEQ ID NO: 53 - protein, 10 aa, synthetic construct
RASSSVSYMY

SEQ ID NO: 54 - protein, 7 aa, synthetic construct
ATSNLAS

SEQ ID NO: 55 - protein, 10 aa, synthetic construct
QQWSSDPPMT

SEQ ID NO: 56 - protein, 447 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNSLLDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 215 aa, synthetic construct
SIVMTQTPKFLLVSAGDRVTITCKASQSVSNAVAWYQQKPGQSPKLLIYYASNRYTGVPDRFTGSGYGTDFTFTISTVQAEDLAVYFCQQDYSSPLTFGAGTKLELKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 243 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNSLLDAMDYWGQGTSVTVSSGGSGGGGSGGGGSGGGGSSIVMTQTPKFLLVSAGDRVTITCKASQSVSNAVAWYQQKPGQSPKLLIYYASNRYTGVPDRFTGSGYGTDFTFTISTVQAEDLAVYFCQQDYSSPLTFGAGTKLELKR

SEQ ID NO: 59 - protein, 446 aa, synthetic construct
QVQLHQPGAELVKPGASVNLSCKASGYTFTSYWMHWVKQRPGQGLEWIGEINPSSGRANYNEKFKNKATLTVDKSSITAYMHLSSLTSEDSAVYYCHWDYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 215 aa, synthetic construct
DIVLTQSPATLSVTPGDSVSLSCRASQSISNNLHWYQQKSHESPRLLFKSASQSISGIPSRFSGSGSGTDFTLSINSVETEDFGMYFCQQTNSWPLTFGTGTKLDLKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 242 aa, synthetic construct
QVQLHQPGAELVKPGASVNLSCKASGYTFTSYWMHWVKQRPGQGLEWIGEINPSSGRANYNEKFKNKATLTVDKSSITAYMHLSSLTSEDSAVYYCHWDYYAMDYWGQGTSVTVSSGGSGGGGSGGGGSGGGGSDIVLTQSPATLSVTPGDSVSLSCRASQSISNNLHWYQQKSHESPRLLFKSASQSISGIPSRFSGSGSGTDFTLSINSVETEDFGMYFCQQTNSWPLTFGTGTKLDLKR

SEQ ID NO: 62 - protein, 446 aa, synthetic construct
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCATFYDGYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 215 aa, synthetic construct
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPFTFGSGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 242 aa, synthetic construct
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCATFYDGYDYWGQGTTLTVSSGGSGGGGSGGGGSGGGGSDIVMTQSHKFMSTSVGDRVSITCKASQDVSTAVAWYQQKPGQSPKLLIYWASTRHTGVPDRFTGSGSGTDYTLTISSVQAEDLALYYCQQHYSTPFTFGSGTKLEIKR

SEQ ID NO: 65 - protein, 449 aa, synthetic construct
QVQLKESGPGLVAPSQSLSISCTVSGFSLTTFGIHWIRQPPGKGLEWLGVIWAAGTTNYNSTLKSRLTITKDNSRSQVFLKMNSLQTYDTAIYYCVREDYDYFFGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 217 aa, synthetic construct
QAVVTQESALTTSPGETVTLTCRSSTGAVTTSNYASWVQEKPDHLFIGLIGGTNNRAPGVPARFSGSLIGDKAALTITGAQTDDEAIYFCALWFSNHWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 247 aa, synthetic construct
QVQLKESGPGLVAPSQSLSISCTVSGFSLTTFGIHWIRQPPGKGLEWLGVIWAAGTTNYNSTLKSRLTITKDNSRSQVFLKMNSLQTYDTAIYYCVREDYDYFFGLDYWGQGTSVTVSSGGSGGGGSGGGGSGGGGSQAVVTQESALTTSPGETVTLTCRSSTGAVTTSNYASWVQEKPDHLFIGLIGGTNNRAPGVPARFSGSLIGDKAALTITGAQTDDEAIYFCALWFSNHWVFGGGTKLTVLG

SEQ ID NO: 68 - protein, 447 aa, synthetic construct
QVQLKESGPGLVAPSQSLSITCTVSGFSLTSYGVHWVRQPPGKGLEWLGVIWADGSTNYNSALMSRLSISKDNSKSQVFLKMNSLQTDDTAMYYCARWTYGDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 215 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 243 aa, synthetic construct
QVQLKESGPGLVAPSQSLSITCTVSGFSLTSYGVHWVRQPPGKGLEWLGVIWADGSTNYNSALMSRLSISKDNSKSQVFLKMNSLQTDDTAMYYCARWTYGDYFDYWGQGTTLTVSSGGSGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 71 - protein, 451 aa, synthetic construct
DVQLVESGGGLVQPGGSRKLSCAASGFTFSYFGMHWVRQAPEKGLEWVAYISSGSNTIYYADTVKGRFTISRDNPKNTLFLQMTSLRSEDTAMYYCARAYYGNHYYAMDFWGPGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 215 aa, synthetic construct
DIQMTQSPASLSVSVGETVTITCRASENIYSNLAWYQQKRGKSPQLLVHAARDLATGVPSRFSGSGSGTQYSLKINSLQSEDFGTYYCQHFWETPWTFGGGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 247 aa, synthetic construct
DVQLVESGGGLVQPGGSRKLSCAASGFTFSYFGMHWVRQAPEKGLEWVAYISSGSNTIYYADTVKGRFTISRDNPKNTLFLQMTSLRSEDTAMYYCARAYYGNHYYAMDFWGPGTSVTVSSGGSGGGGSGGGGSGGGGSDIQMTQSPASLSVSVGETVTITCRASENIYSNLAWYQQKRGKSPQLLVHAARDLATGVPSRFSGSGSGTQYSLKINSLQSEDFGTYYCQHFWETPWTFGGGTKLEIKR

SEQ ID NO: 74 - protein, 446 aa, synthetic construct
EVQLVESGGGLVKPGGSLKLSCAASGFTFSDYYMYWIRQTPEKRLEWVATISDGGFSTYYPDSVKGRFTISRDNAKNNLFLQMSSLKSEDTAIYYCARFGAYSTFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 215 aa, synthetic construct
QIVLSQSPAILSASPGEKVTMTCRASSSVSYMYWYQQKPGSSPKPWIYATSNLASGVPARFSGSGSGTSYSLTISRVEAEDAATYYCQQWSSDPPMTFGGGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 242 aa, synthetic construct
EVQLVESGGGLVKPGGSLKLSCAASGFTFSDYYMYWIRQTPEKRLEWVATISDGGFSTYYPDSVKGRFTISRDNAKNNLFLQMSSLKSEDTAIYYCARFGAYSTFWGQGTLVTVSSGGSGGGGSGGGGSGGGGSQIVLSQSPAILSASPGEKVTMTCRASSSVSYMYWYQQKPGSSPKPWIYATSNLASGVPARFSGSGSGTSYSLTISRVEAEDAATYYCQQWSSDPPMTFGGGTKLEIKR

SEQ ID NO: 77 - protein, 18 aa, synthetic construct
GGSGGGGSGGGGSGGGGS

SEQ ID NO: 78 - DNA, 36 bp, synthetic construct
  source 1..36: mol_type = other DNA
atgaattattttgatactgaggaagattacttggaa

SEQ ID NO: 79 - DNA, 33 bp, synthetic construct
  source 1..33: mol_type = other DNA
ctttcttgagaaatagataccaaagtacttcag

SEQ ID NO: 80 - DNA, 20 bp, synthetic construct
  source 1..20: mol_type = other DNA
cgaaagcatttgccaagaat

SEQ ID NO: 81 - DNA, 20 bp, synthetic construct
  source 1..20: mol_type = other DNA
gcatcgtttatggtcggaac

SEQ ID NO: 82 - protein, 9 aa, synthetic construct
DKKVEPKSC

(19) United States
(12) Patent Application Publication
(10) Pub. No.: US 2024/0391980 A1
(43) Pub. Date: Nov. 28, 2024

(54) Anti-Human Astrovirus Antibodies and Related Compositions and Methods

(71) Applicants: The Regents of the University of California, Oakland, CA (US); Universidad Nacional Autónoma de México, Ciudad de México (MX)

(72) Inventors: Rebecca Dubois, Santa Cruz, CA (US); Lena Ricemeyer, Santa Cruz, CA (US); Carlos F. Arias Ortiz, Cuernavaca, Morelos (MX); Tomás López Díaz, Cuernavaca, Morelos (MX); Rafaela Espinosa Organista, Cuernavaca, Morelos (MX)

(21) Appl. No.: 18/696,922

(22) PCT Filed: Sep. 30, 2022
(86) PCT No.: PCT/US2022/077434

§ 371 Date: Mar. 28, 2024

Related U.S. Application Data
(60) Provisional application No. 63/250,551, filed on Sep. 30, 2021.

(51) Int. Cl. C07K 16/10; A61P 31/14

(52) U.S. Cl. CPC C07K 16/10; A61P 31/14; C07K 2317/24; C07K 2317/31; C07K 2317/52; C07K 2317/565; C07K 2317/622; C07K 2317/76

(57) ABSTRACT

Provided are antibodies that specifically bind human astrovirus (HAstV) capsid spike protein. Fusion proteins and conjugates comprising such antibodies are also provided. Also provided are nucleic acids that encode one or both of the variable chain polypeptides of an antibody of the present disclosure, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual having or suspected of having a HAstV infection a therapeutically effective amount of an antibody, fusion protein or conjugate of the present disclosure.

### CROSS-REFERENCE TO RELATED APPLICATIONS

This application claims the benefit of U.S. Provisional Patent Application No. 63/250,551, filed Sep. 30, 2021, which application is incorporated herein by reference in its entirety.

### STATEMENT OF GOVERNMENT SUPPORT

This invention was made with Government support under Grant No. A1144090, awarded by the National Institutes of Health (NIH). The Government has certain rights in the invention.

### INTRODUCTION

Astroviruses are a distinct family of small, non-enveloped RNA viruses that infect mammalian and avian species.¹ Members of the Avastrovirus genus have been associated with a variety of disease manifestations, growth defects, and mortality in poultry.² Members of the Mamastrovirus genus cause infections in humans and a wide range of other mammals, indicating the potential for a zoonotic disease transmission and the emergence of new astrovirus strains that could threaten human health.³ Within Mamastrovirus, human astroviruses (HAstVs) are classified into eight canonical serotypes (HAstV1-8), where serotype 1 is the most prevalent globally.⁴⁻⁷ HAstV is a leading worldwide cause of viral gastroenteritis but remains one of the most inadequately understood enteric viruses.⁸ In particular, young children, the elderly, and the immunocompromised are at risk for astrovirus infection, especially in developing countries.⁹⁻¹⁶ The United States alone reports approximately 3.9 million cases of HAstV gastroenteritis each year,¹⁷ and children under 12 months may require hospitalization.¹⁸ While HAstV infection accounts for an estimated 2 to 9% of all acute non-bacterial gastroenteritis in healthy children worldwide,¹⁹ studies found that HAstV also causes persistent infections that spread easily in pediatric oncology wards.^{20, 21} In addition to the canonical serotypes causing gastroenteritis, highly divergent MLB and VA clades have recently been associated with neurological complications such as encephalitis in immunocompromised and immunocompetent individuals, demonstrating that astroviruses not only infect cells in the gastrointestinal tract but also have systemic potential.²²⁻²⁵ However, no vaccine preventing human astrovirus infection has been developed. Additionally, while a few existing antiviral small molecule therapies have been shown to be effective against multiple astrovirus serotypes in vitro and in the turkey poult animal model, their use to treat human astrovirus infection in humans has yet to be reported.

A growing body of evidence underlines the importance of antibodies in protecting healthy adults from infection. Firstly, human astrovirus infection is rare in adults, indicating that a protective adaptive immune response conveying lifelong immunity develops during childhood.²⁶ In fact, greater than 75% of healthy adults have anti-HAstV antibodies targeting at least one of the eight classical serotypes,^{7, 26} and seroprevalence rates increase with age.⁵ Secondly, clinical studies determined that more severe disease is correlated with a lack of anti-HAstV antibodies in healthy volunteers.^{27, 28} Finally, immunoglobulin replacement therapy facilitated the recovery of an immunocompromised patient from persistent HAstV infection.²⁹ Together, these findings suggest that the incidence of HAstV gastroenteritis is likely underappreciated. In addition, these observations reveal that the adaptive immune response plays a crucial role in shielding an individual from HAstV disease. Accordingly, a vaccine eliciting protective antibodies would likely reduce HAstV infection in vulnerable populations. However, rational design of subunit vaccine immunogens or antiviral therapies relies on an understanding of the sites at which neutralizing antibodies bind human astrovirus and on insight into viral defenses against antibody neutralization.

HAstV particles contain a 6-7 kb, positive-sense, single-stranded RNA genome surrounded by a ˜35 nm non-enveloped capsid protein shell. The genome's three open reading frames (ORFs) encode non-structural polyproteins (ORF1a and ORF1b) and the multi-domain capsid protein (ORF2).^{30, 31} This capsid protein contains a highly basic N-terminal region, a core domain, a spike domain, and a C-terminal acidic region.³² During maturation, HAstV capsid proteins undergo a series of intra- and extracellular proteolytic cleavages that are required for infectivity.³³⁻³⁶ The capsid core domain forms the T=3 icosahedral shell that encapsidates the viral RNA genome.^{37, 38} The capsid spike domain forms the thirty dimeric spike projections on the surface of the mature virus particle.^{37, 39, 40} Only one neutralizing epitope, located on the capsid spike domain, has been defined by X-ray crystallography of an antibody/spike complex.⁴⁰ This study provided evidence that the spike is a receptor-binding domain and that this antibody, PL-2, neutralizes HAstV2 by obstructing a receptor-binding site on the spike. Further study has shown that while both the core and spike domains of the HAstV capsid are immunogenic, only the spike domain elicits antibodies that neutralize virus infectivity.⁴¹ Indeed, the neutralizing monoclonal antibodies against HAstV for which neutralization mechanisms have been described all target the spike domain.⁴¹⁻⁴³

### SUMMARY

Provided are antibodies that specifically bind human astrovirus (HAstV) capsid spike protein. Fusion proteins and conjugates comprising such antibodies are also provided. Also provided are nucleic acids that encode one or both of the variable chain polypeptides of an antibody of the present disclosure, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual having or suspected of having a HAstV infection a therapeutically effective amount of an antibody, fusion protein or conjugate of the present disclosure.

### BRIEF DESCRIPTION OF THE FIGURES

FIG. 1 scFv 3E8 and scFv 2D9 bind Spike 8 with high affinity. (A) Affinity determination for the scFv 3E8/Spike 8 interaction. (B) Affinity determination for the scFv 2D9/Spike 8 interaction. (C) Competition BLI assay showing simultaneous binding of scFvs 3E8 and 2D9. Data shown in 1A and 1 B are from one representative experiment of the triplicate assays.

FIG. 2 Intermolecular interactions at the scFv 3E8/Spike 8 interface. (A) Semitransparent surface representation of the scFv 3E8/Spike 8 complex. Shown are the Spike 8 dimer, scFv 3E8 kappa chain, and scFv 3E8 heavy chain. Primary regions of interaction are highlighted by black boxes with labels that correspond to the zoomed-in views in panels B and C. Dashed lines represent hydrogen bonds. Escape mutant residue Y464 is indicated by a box around the residue name. (B) Interactions of 3E8 CDRs K3, H1, H2, and H3 with Spike 8 Loop 1. Interactions of 3E8 CDRs K1, K2, K3, and H3 with the Spike 8 β8 strand. Interactions of CDRs K1 and K3 with the Spike 8 β9 strand. (C) Interactions of 3E8 CDRs H1 and H3 with Spike 8 β5-β6 strands.

FIG. 3 Intermolecular interactions at the scFv 2D9/Spike 8 interface. (A) Semitransparent surface representation of the scFv 2D9/Spike 8 complex. Shown are the Spike 8 dimer, scFv 2D9 kappa chain, and scFv 2D9 heavy chain. Primary regions of interaction are highlighted by black boxes with labels that correspond to the zoomed-in views in panels B and C. Dashed lines represent hydrogen bonds. Escape mutant residue D597 is indicated by a box around the residue name. (B) Interactions of 2D9 CDRs K1 and K2 with Spike 8 Loop 1 and Spike 8 Loop 3. (C) Interactions of 2D9 CDRs K2, K3, H2, and H3 with Spike 8 β6-β7 and β10 strands as well as Loop 4.

FIG. 4 Comparison of epitopes for 3E8/Spike 8, 2D9/Spike 8, and PL-2/Spike 2 in relation to the P site. (A) Structure of scFv 3E8, scFv 2D9, and PL-2 bound to spike viewed from the side and from the top. (B) Epitope footprints on spike. (C) Sequence alignment of the capsid spike domains of HAstV1-8. Conserved, strongly similar, weakly similar, and non-conserved amino acids are on a gradient where darker color represents more conserved. Alignments were done with Clustal Omega,⁴⁴ and mapping of conservation onto the structure was performed with the online ESPript server.⁴⁵ Dots below the sequence alignment indicate residues in the epitopes targeted by 3E8, 2D9, and PL-2 as well as residues in the P site.

FIG. 5 Antibodies 3E8 and 2D9 reduce virus infectivity, block virus attachment, and detach the virus from Caco-2 cells. (A) 3E8 and 2D9 reduce virus infectivity. For each dilution, 3E8 and 2D9 are provided from left to right. (B) 3E8 and 2D9 block virus attachment when HAstV8 is pre-incubated with the antibodies. For each dilution, 3E8, 2D9 and 3B4 are provided from left to right. (C) 3E8 and 2D9 detach HAstV8 from cells when the antibodies are added after the virus has attached. For each dilution, 3E8, 2D9 and 3B4 are provided from left to right.

FIG. 6 Antibodies 3E8 and 2D9 block GFP-Spike 8 attachment to Caco-2 cells. GFP fluorescence is green and Hoechst DNA stain is blue. (A) Caco-2 cells incubated with PBS show slight autofluorescence. (B) Caco-2 cells incubated with GFP alone show little to no non-specific GFP binding. (C) Caco-2 cells incubated with GFP-Spike 8 show bright punctate patterns at or near the cell membranes, suggesting spike-specific attachment. (D) Fab 3E8 blocks GFP-Spike 8 attachment to Caco-2 cells. (E) Fab 2D9 blocks GFP-Spike 8 attachment to Caco-2 cells. (F) Fab 3B4 does not block GFP-Spike 8 attachment to Caco-2 cells.

### DETAILED DESCRIPTION

Before the antibodies, compositions and methods of the present disclosure are described in greater detail, it is to be understood that the antibodies, compositions and methods are not limited to particular embodiments described, as such may, of course, vary. It is also to be understood that the terminology used herein is for the purpose of describing particular embodiments only, and is not intended to be limiting, since the scope of the antibodies, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies, compositions and methods. The upper and lower limits of these smaller ranges may independently be included in the smaller ranges and are also encompassed within the antibodies, compositions and methods, subject to any specifically excluded limit in the stated range. Where the stated range includes one or both of the limits, ranges excluding either or both of those included limits are also included in the antibodies, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies, compositions and methods belong. Although any antibodies, compositions and methods similar or equivalent to those described herein can also be used in the practice or testing of the antibodies, compositions and methods, representative illustrative antibodies, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies, compositions and methods, which are, for clarity, described in the context of separate embodiments, may also be provided in combination in a single embodiment. Conversely, various features of the antibodies, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Antibodies

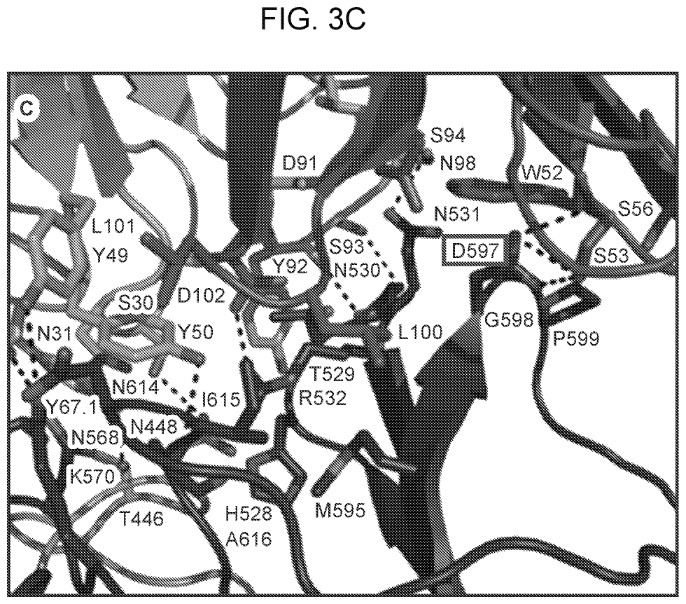

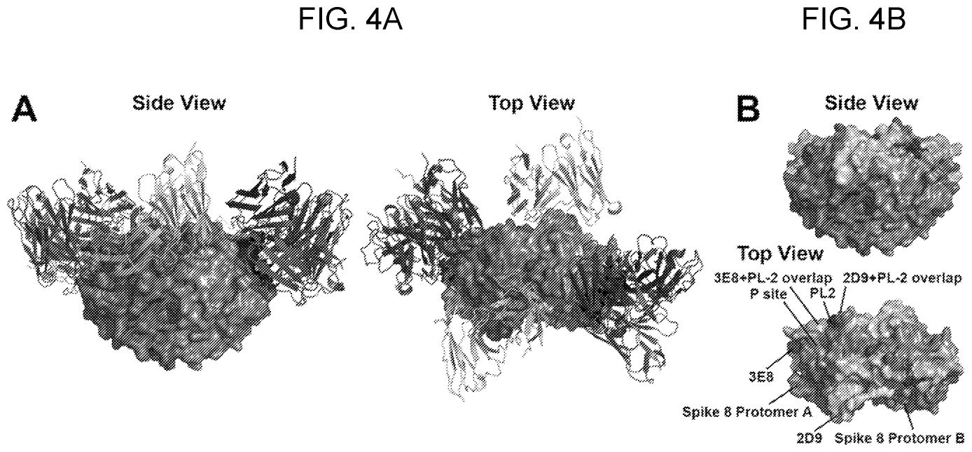

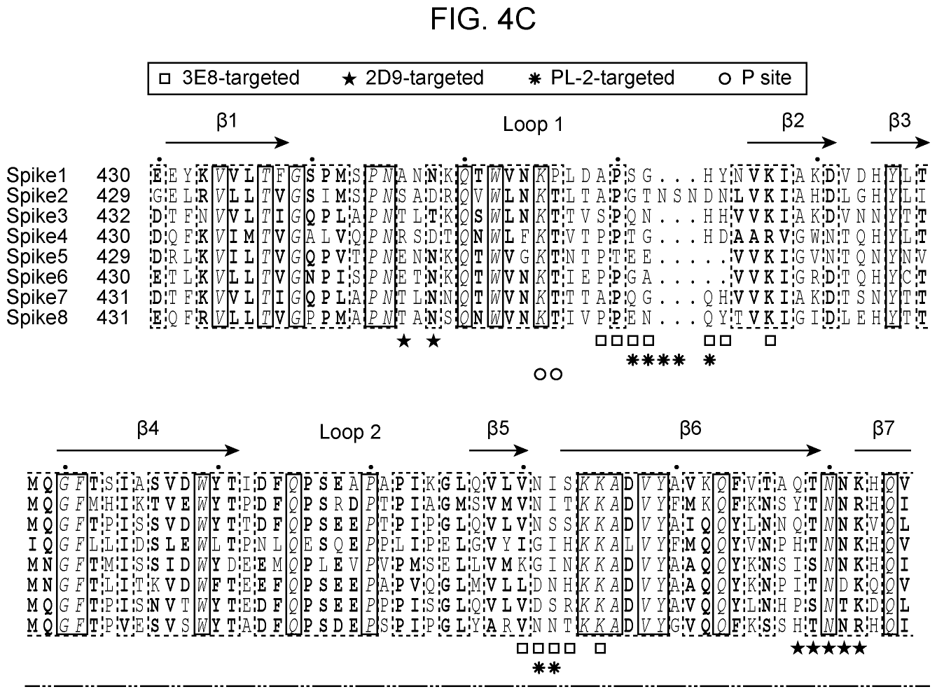

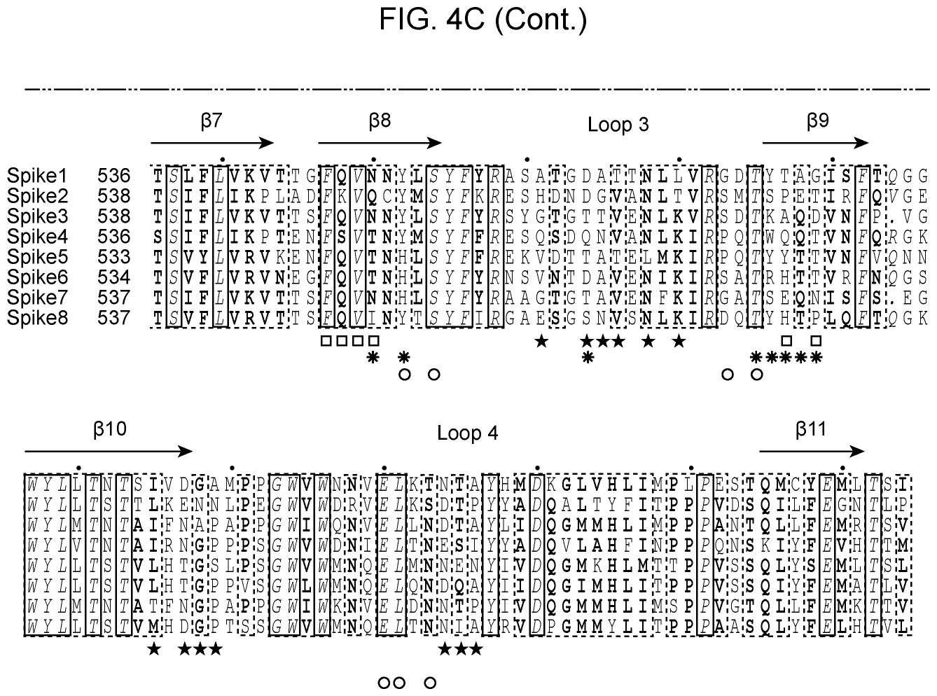

The present disclosure provides antibodies that specifically bind to a human astrovirus (HAstV) capsid spike protein. In certain embodiments, an antibody of the present disclosure specifically binds to a HAstV capsid spike protein of HAstV serotype 1 (spike 1), a HAstV capsid spike protein of HAstV serotype 2 (spike 2), a HAstV capsid spike protein of HAstV serotype 3 (spike 3), a HAstV capsid spike protein of HAstV serotype 4 (spike 4), a HAstV capsid spike protein of HAstV serotype 5 (spike 5), a HAstV capsid spike protein of HAstV serotype 6 (spike 6), a HAstV capsid spike protein of HAstV serotype 7 (spike 7), a HAstV capsid spike protein of HAstV serotype 8 (spike 8), or any combination thereof. The amino acid sequences of such spike proteins are provided in FIG. 4, panel C.

The term “antibody” (also used interchangeably with “immunoglobulin”) encompasses polyclonal (e.g., rabbit polyclonal) and monoclonal antibody preparations where the antibody may be an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein. In some embodiments, the antibody is selected from an IgG, Fv, single chain antibody, scFv, a Fab, a F(ab′)₂, and a F(ab′). The antibodies may be further conjugated to other moieties, such as members of specific binding pairs, e.g., biotin (member of biotin-avidin specific binding pair), and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics Information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, an antibody of the present disclosure comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH-CHI by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies. In certain embodiments, an antibody of the present disclosure is selected from an IgG, Fv, single chain antibody, scFv, Fab, F(ab′)₂, and Fab′.

According to some embodiments, an antibody of the present disclosure is a monoclonal antibody. “Monoclonal antibody” refers to a composition comprising one or more antibodies obtained from a population of substantially homogeneous antibodies, i.e., a population the individual antibodies of which are identical except for any naturally occurring mutations that may be present in minor amounts. Monoclonal antibodies are highly specific, being directed against a single antigenic site and generally to a single epitope on an antigen. The modifier “monoclonal” indicates the character of the antibody as being obtained from a substantially homogeneous population of antibodies, and does not require that the antibody be produced by any particular method or be the only antibody in the composition.

In certain embodiments, an antibody of the present disclosure is a humanized antibody. As used herein, a humanized antibody is a recombinant polypeptide that is derived from a non-human (e.g., rabbit, rodent, or the like) antibody and has been modified to contain at least a portion of the framework and/or constant regions of a human antibody. Humanized antibodies also encompass chimeric antibodies and CDR-grafted antibodies in which various regions may be derived from different species. Chimeric antibodies may be antibodies that include a variable region from any source linked to a human constant region (e.g., a human Fc domain). Thus, in chimeric antibodies, the variable region can be non-human, and the constant region is human. CDR-grafted antibodies are antibodies that include the CDRs from a non-human “donor” antibody linked to the framework region from a human “recipient” antibody. For example, an antibody of the present disclosure in a form of an scFV may be linked to a human constant region (e.g., Fc domain) to be made into a human immunoglobulin.

In general, humanized antibodies produce a reduced immune response in a human host, as compared to a non-humanized version of the same antibody. Antibodies can be humanized using a variety of techniques including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions.

Accordingly, any of the antibodies described herein may be humanized using available methods. The substitution of rabbit or mouse CDRs into a human variable domain framework can result in retention of their correct spatial orientation where, e.g., the human variable domain framework adopts the same or similar conformation to the rabbit or mouse variable framework from which the CDRs originated. This can be achieved by obtaining the human variable domains from human antibodies whose framework sequences exhibit a high degree of sequence identity with the rabbit or mouse variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the rabbit or mouse donor immunoglobulin and appropriate human acceptor immunoglobulins, the next step is to determine which, if any, residues from these components should be substituted to optimize the properties of the resulting humanized antibody. In general, substitution of human amino acid residues with rabbit or mouse should be minimized, because introduction of rabbit or mouse residues increases the risk of the antibody eliciting a human-anti-rabbit-antibody (HARA) or human-anti-mouse-antibody (HAMA) response in humans. Art-recognized methods of determining immune response can be performed to monitor a HARA or HAMA response in a particular patient or during clinical trials. Patients administered humanized antibodies can be given an immunogenicity assessment at the beginning and throughout the administration of said therapy. The HARA or HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HARA response in a human subject.

Certain amino acids from the human variable region framework residues are selected for substitution based on their possible influence on CDR conformation and/or binding to antigen. The unnatural juxtaposition of rabbit or murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by, e.g., Kabat, constitute structural loop residues as defined by, e.g., Chothia, the amino acids present in the rabbit or mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody. Approaches that may be employed to humanize any of the antibodies described herein include, but are not limited to, those described in Williams, D., Matthews, D. & Jones, T. Humanising Antibodies by CDR Grafting. Antibody Engineering 319-339 (2010) doi:10.1007/978-3-642-01144-3_21; Kuramochi, T., Igawa, T., Tsunoda, H. & Hattori, K. Humanization and simultaneous optimization of monoclonal antibody. Methods Mol. Biol. 1060,123-37 (2014); Hwang, W. Y., Almagro, J. C., Buss, T. N., Tan, P. & Foote, J. Use of human germline genes in a CDR homology-based approach to antibody humanization. Methods 36, 35-42 (2005); Lo, B. K. Antibody humanization by CDR grafting. Methods Mol. Biol. 248, 135-59 (2004); and Lefranc, M.-P. P., Ehrenmann, F., Ginestoux, C., Giudicelli, V. & Duroux, P. Use of IMGT(®) databases and tools for antibody engineering and humanization. Methods Mol. Biol. 907, 3-37 (2012); the disclosures of which are incorporated herein by reference in their entireties for all purposes.

An antibody of the present disclosure specifically binds to a HAstV capsid spike protein. An antibody “specifically binds” or “preferentially binds” to a target if it binds with greater affinity, avidity, more readily, and/or with greater duration than it binds to other substances, e.g., in a sample. In certain embodiments, an antibody “specifically binds” an antigen if it binds to or associates with the antigen with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means the antibody binds to the antigen with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the antigen can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

Whether an antibody of the present disclosure “competes with” a second antibody for binding to the antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labeled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 50% or more, about 60% or more, about 70% or more, about 80% or more, about 85% or more, about 90% or more, about 95% or more, or about 99% or more. Details of procedures for carrying out such competition assays are known and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

In certain embodiments, an antibody of the present disclosure specifically binds HAstV capsid spike protein and competes for binding to the HAstV capsid spike protein with an antibody comprising one, two, three, four, five, or all six complementarity determining regions (CDRs) of one or more of the anti-HAstV capsid spike protein antibodies designated herein as 2D9, 3B4, 3E8, 3H4, 4B6, 2A2, and 7C8. According to some embodiments, an antibody of the present disclosure specifically binds HAstV capsid spike protein and comprises one, two, three, four, five, or all six CDRs of the anti-HAstV capsid spike protein antibody designated herein as 2D9, 3B4, 3E8, 3H4, 4B6, 2A2, or 7C8. The amino acid sequences of the variable heavy chain (V_{H}) polypeptides, the variable light chain (V_{L}) polypeptides, and the CDRs of the 2D9, 3B4, 3E8, 3H4, 4B6, 2A2, and 7C8 antibodies are provided in Table 3 below. All CDRs and framework regions described throughout the present disclosure are defined according to Kabat, supra, unless otherwise indicated.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GFSLTSYGVH (SEQ ID NO:2),
   a V_{H} CDR2 comprising the amino acid sequence VIWSGGSTDYNAAFIS (SEQ ID NO:3), and
   a V_{H} CDR3 comprising the amino acid sequence NSLLDAMDY (SEQ ID NO:4), and
a variable light chain (V_{L}) polypeptide comprising
   a V_{L} CDR1 comprising the amino acid sequence KASQSVSNAVA (SEQ ID NO:6),
   a V_{L} CDR2 comprising the amino acid sequence YASNRYT (SEQ ID NO:7), and
   a V_{L} CDR3 comprising the amino acid sequence QQDYSSPLT (SEQ ID NO:8).

In certain embodiments, such an antibody comprises one, two, three, four, five, or all six 35 CDRs set forth in SEQ ID NO:2, SEQ ID NO:3, SEQ ID NO:4, SEQ ID NO:6, SEQ ID NO:7, and SEQ ID NO:8. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:1; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:5; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GYTFTSYWMH (SEQ ID NO:10),
   a V_{H} CDR2 comprising the amino acid sequence EINPSSGRANYNEKFKN (SEQ ID NO:11), and
   a VH CDR3 comprising the amino acid sequence DYYAMDY (SEQ ID NO:12), and
a variable light chain (V_{L}) polypeptide comprising
   a V_{L} CDR1 comprising the amino acid sequence RASQSISNNLH (SEQ ID NO:14),
   a V_{L} CDR2 comprising the amino acid sequence SASQSIS (SEQ ID NO:15), and
   a V_{L} CDR3 comprising the amino acid sequence QQTNSWPLT (SEQ ID NO:16).

In certain embodiments, such an antibody comprises one, two, three, four, five, or all six CDRs set forth in SEQ ID NO:10, SEQ ID NO:11, SEQ ID NO:12, SEQ ID NO:14, SEQ ID NO:15, and SEQ ID NO:16. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:9; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:13; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GYSITSGYYWN (SEQ ID NO:18),
   a V_{H} CDR2 comprising the amino acid sequence YISYDGSNNYNPSLKN (SEQ ID NO:19), and
   a V_{H} CDR3 comprising the amino acid sequence FYDGYDY (SEQ ID NO:20), and
a variable light chain (V_{L}) polypeptide comprising
   a V_{L} CDR1 comprising the amino acid sequence KASQDVSTAVA (SEQ ID NO:22),
   a V_{L} CDR2 comprising the amino acid sequence WASTRHT (SEQ ID NO:23), and
   a V_{L} CDR3 comprising the amino acid sequence QQHYSTPFT (SEQ ID NO:24).

In certain embodiments, such an antibody comprises one, two, three, four, five, or all six CDRs set forth in SEQ ID NO:18, SEQ ID NO:19, SEQ ID NO:20, SEQ ID NO:22, SEQ ID NO:23, and SEQ ID NO:24. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:17; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:21; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GFSLTTFGIH (SEQ ID NO:26),
   a V_{H} CDR2 comprising the amino acid sequence VIWAAGTTNYNSTLKS (SEQ ID NO:27), and
   a V_{H} CDR3 comprising the amino acid sequence EDYDYFFGLDY (SEQ ID NO:28), and
a variable light chain (V_{L}) polypeptide comprising
   a V_{L} CDR1 comprising the amino acid sequence RSSTGAVTTSNYAS (SEQ ID NO:30),
   a V_{L} CDR2 comprising the amino acid sequence GTNNRAP (SEQ ID NO:31), and
   a V_{L} CDR3 comprising the amino acid sequence ALWFSNHWV (SEQ ID NO:32).

In certain embodiments, such an antibody comprises one, two, three, four, five, or all six CDRs set forth in SEQ ID NO:26, SEQ ID NO:27, SEQ ID NO:28, SEQ ID NO:30, SEQ ID NO:31, and SEQ ID NO:32. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:25; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:29; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GFSLTSYGVH (SEQ ID NO:1),
   a V_{H} CDR2 comprising the amino acid sequence VIWADGSTNYNSALMS (SEQ ID NO:34), and
   a V_{H} CDR3 comprising the amino acid sequence WTYGDYFDY (SEQ ID NO:35), and
a variable light chain (V_{L}) polypeptide comprising
   a V_{L} CDR1 comprising the amino acid sequence SASQGISNYLN (SEQ ID NO:37),
   a V_{L} CDR2 comprising the amino acid sequence YTSSLHS (SEQ ID NO:38), and
   a V_{L} CDR3 comprising the amino acid sequence QQYSKLPYT (SEQ ID NO:39).

In certain embodiments, such an antibody comprises one, two, three, four, five, or all six CDRs set forth in SEQ ID NO:1, SEQ ID NO:34, SEQ ID NO:35, SEQ ID NO:37, SEQ ID NO:38, and SEQ ID NO:39. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:33; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:36; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GFTFSYFGMH (SEQ ID NO:41),
   a V_{H} CDR2 comprising the amino acid sequence YISSGSNTIYYADTVKG (SEQ ID NO:42), and
   a V_{H} CDR3 comprising the amino acid sequence AYYGNHYYAMDF (SEQ ID NO:43), and
a variable light chain (V_{L}) polypeptide comprising
   a V_{L} CDR1 comprising the amino acid sequence RASENIYSNLA (SEQ ID NO:45),
   a V_{L} CDR2 comprising the amino acid sequence AARDLAT (SEQ ID NO:46), and
   a VL CDR3 comprising the amino acid sequence QHFWETPWT (SEQ ID NO:47).

In certain embodiments, such an antibody comprises one, two, three, four, five, or all six CDRs set forth in SEQ ID NO:41, SEQ ID NO:42, SEQ ID NO:43, SEQ ID NO:45, SEQ ID NO:46, and SEQ ID NO:47. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:40; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:44; or both.

In certain embodiments, an antibody of the present disclosure specifically binds a HAstV capsid spike protein and competes for binding to the HAstV capsid spike protein with an antibody comprising:
a variable heavy chain (V_{H}) polypeptide comprising
   a V_{H} CDR1 comprising the amino acid sequence GFTFSDYYMY (SEQ ID NO:49),
   a V_{H} CDR2 comprising the amino acid sequence TISDGGFSTYYPDSVKG (SEQ ID NO:50), and
   a V_{H} CDR3 comprising the amino acid sequence FGAYSTF (SEQ ID NO:51), and
a variable light chain (V_{L}) polypeptide comprising
   a V_{L} CDR1 comprising the amino acid sequence RASSSVSYMY (SEQ ID NO:53),
   a V_{L} CDR2 comprising the amino acid sequence ATSNLAS (SEQ ID NO:54), and
   a V_{L} CDR3 comprising the amino acid sequence QQWSSDPPMT (SEQ ID NO:55).

In certain embodiments, such an antibody comprises one, two, three, four, five, or all six CDRs set forth in SEQ ID NO:49, SEQ ID NO:50, SEQ ID NO:51, SEQ ID NO:53, SEQ ID NO:54, and SEQ ID NO:55. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:48; a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:52; or both.

### Bispecific Antibodies

Also provided are bispecific antibodies. In certain embodiments, a bispecific antibody of the present disclosure comprises a first antigen-binding domain comprising a V_{H} polypeptide-V_{L}polypeptide pair of any of the anti-HAstV capsid spike protein antibodies of the present disclosure, including any of such antibodies described elsewhere herein, e.g., any of the antibodies having the amino acid sequences provided in Table 3 below. The bispecific antibody may include a second antigen-binding domain that specifically binds the HAstV capsid spike protein bound by the first antigen-binding domain. In certain embodiments, the bispecific antibody includes a second antigen-binding domain that specifically binds an antigen other than the HAstV capsid spike protein bound by the first antigen-binding domain.

Bispecific antibodies of the present disclosure include antibodies having a full-length antibody structure, and bispecific antibody fragments. “Full-length” as used herein refers to an antibody having two full-length antibody heavy chains and two full length antibody light chains. A full-length antibody heavy chain (HC) consists of well-known heavy chain variable and constant domains VH, CH1, CH2, and CH3. A full-length antibody light chain (LC) consists of well-known light chain variable and constant domains VL and CL. The full-length antibody may be lacking the C-terminal lysine in either one or both heavy chains. The term “Fab arm” refers to one heavy chain:light chain pair that specifically binds an antigen.

Full-length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in a cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US2010/0015133; US2009/0182127; US2010/028637 or US2011/0123532. In other strategies. heterodimerization may be promoted by the following substitutions (expressed as modified position in the first CH3 domain of the first heavy chain/modified position in the second CH3 domain of the second heavy chain): L351 Y_F405A_Y407V T394W, T366I_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8(18):3689-3696.

Approaches that may be employed to produce multispecific (e.g., bispecific) antibodies from the antibodies described herein include, but are not limited to, Ellerman, D. (2019). “Bispecific T-cell engagers: Towards understanding variables influencing the in vitro potency and tumor selectivity and their modulation to enhance their efficacy and safety.” Methods 154: 102-117; Brinkmann, U. and R. E. Kontermann (2017). “The making of bispecific antibodies.” mAbs 9(2): 182-212; and Suurs, F. V., et al. (2019). “A review of bispecific antibodies and antibody constructs in oncology and clinical challenges.” Pharmacol Ther 201: 103-119; the disclosures of which are incorporated herein by reference in their entireties for all purposes.

### Fusion Proteins

Also provided are fusion proteins. In certain embodiments, a fusion protein of the present disclosure comprises a chain of any of the anti-HAstV capsid spike protein antibodies of the present disclosure (including any of such antibodies described elsewhere herein, e.g., any of the antibodies having the amino acid sequences provided in Table 3 below), fused to a heterologous sequence of amino acids. The heterologous sequence of amino acids may be fused to the C-terminus of the chain of the antibody or the N-terminus of the chain of the antibody. In certain embodiments, a fusion protein of the present disclosure includes a heterologous sequence at the C-terminus of the chain of the antibody and a heterologous sequence at the N-terminus of the chain of the antibody, wherein the heterologous sequences may be the same sequence or different sequences. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, red fluorescent protein (e.g., mCherry), luciferase, etc.) are said to be “heterologous” to one another. Similarly, a CDR from a mouse antibody and a constant region from a human antibody are “heterologous” to one another.

The chain of the anti-HAstV capsid spike protein antibody may be fused to any heterologous sequence of interest. Heterologous sequences of interest include, but are not limited to, an albumin, a transferrin, XTEN, a homo-amino acid polymer, a proline-alanine-serine polymer, an elastin-like peptide, or any combination thereof. In certain aspects, the heterologous polypeptide increases the stability and/or serum half-life of the antibody upon its administration to an individual in need thereof, as compared to the same antibody which is not fused to the heterologous sequence.

In certain embodiments, a fusion protein of the present disclosure comprises a single chain antibody, e.g., a single chain antibody (e.g., scFv) comprising a V_{H} polypeptide-V_{L}polypeptide pair of any of the anti-HAstV capsid spike protein antibodies of the present disclosure, including any of such antibodies described elsewhere herein, e.g., any of the antibodies having the amino acid sequences provided in Table 3 below. scFvs of the present disclosure include, but are not limited to, scFvs comprising the six CDRs of an scFv set forth in Table 3 below, which scFv in some embodiments comprises a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence of the V_{H} of the scFv set forth in Table 3; and a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence of the V_{L} of the scFv set forth in Table 3.

### Conjugates

The present disclosure also provides conjugates. According to some embodiments, a conjugate of the present disclosure comprises any of the antibodies or fusion proteins of the present disclosure, and an agent conjugated to the antibody or fusion protein. The term “conjugated” generally refers to a chemical linkage, either covalent or non-covalent, usually covalent, that proximally associates one molecule of interest with a second molecule of interest. In certain embodiments, the agent conjugated to the antibody or fusion protein is a detectable label or a half-life extending moiety.

According to some embodiments, the agent is a labeling agent. By “labeling agent” (or “detectable label”) is meant the agent detectably labels the antibody or fusion protein, such that the antibody or fusion protein may be detected in an application of interest (e.g., in vitro and/or in vivo research and/or clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain aspects, the antibody or fusion protein is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 8000W Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

For half-life extension, the antibodies and fusion proteins of the present disclosure may be conjugated to an agent that provides for an improved pharmacokinetic profile (e.g., by PEGylation, hyperglycosylation, and the like). Modifications that can enhance serum half-life are of interest. A subject antibody or fusion protein may be “PEGylated”, as containing one or more poly(ethylene glycol) (PEG) moieties. Methods and reagents suitable for PEGylation of a protein are well known in the art and may be found, e.g., in U.S. Pat. No. 5,849,860. PEG suitable for conjugation to a protein is generally soluble in water at room temperature and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody or fusion protein can be linear. The PEG conjugated to the subject antibody or fusion protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody or fusion protein is to be isolated from a source, the antibody or fusion protein may be conjugated to one or more moieties that facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies or fusion proteins are to be detected in an assay, the antibodies or fusion proteins may contain a detectable label, e.g., a radioisotope (e.g., ⁸⁹Zr; ¹¹¹In, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., ¹⁵²Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

Any of the above agents may be conjugated to the antibody or fusion protein via a linker. If present, the linker molecule(s) may be of sufficient length to permit the antibody or fusion protein and the linked agent to allow some flexible movement between the antibody or fusion protein and the linked agent. Linker molecules may be, e.g., about 6-50 atoms long. Linker molecules may also be, e.g., aryl acetylene, ethylene glycol oligomers containing 2-10 monomer units, diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of an antibody or fusion protein conjugated to any agents described above can include linkers that are all or partially flexible, such that the linker can include a flexible linker as well as one or more portions that confer a less flexible structure.

According to some embodiments, the antibody or fusion protein is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9(1):33-46; and elsewhere.

Numerous strategies are available for linking agents to an antibody or fusion protein directly, or indirectly via a linker. For example, the agent may be derivatized by covalently attaching a linker to the agent, where the linker has a functional group capable of reacting with a “chemical handle” on the antibody or fusion protein. The functional group on the linker may vary and may be selected based on compatibility with the chemical handle on the antibody or fusion protein. According to one embodiment, the chemical handle on the antibody or fusion protein is provided by incorporation of an unnatural amino acid having the chemical handle into the antibody or fusion protein. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. An unnatural amino acid may be incorporated into an antibody or fusion protein via chemical synthesis or recombinant approaches, e.g., using a suitable orthogonal amino acyl tRNA synthetase-tRNA pair for incorporation of the unnatural amino acid during translation of the antibody or fusion protein in a host cell.

The functional group of an unnatural amino acid present in the antibody or fusion protein may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody or fusion protein and the linker portion of a linker-agent moiety may include an alkyne functional group, such that the antibody or fusion protein and linker-agent moiety are covalently conjugated via azide-alkyne cycloaddition. Conjugation may be carried out using, e.g., a copper-catalyzed azide-alkyne cycloaddition reaction.

In certain embodiments, the chemical handle on the antibody or fusion protein does not involve an unnatural amino acid. An antibody containing no unnatural amino acids may be conjugated to the agent by utilizing, e.g., nucleophilic functional groups of the antibody or fusion protein (such as the N-terminal amine or the primary amine of lysine, or any other nucleophilic amino acid residue) as a nucleophile in a substitution reaction with a moiety bearing a reactive leaving group or other electrophilic group. An example would be to prepare an agent-linker moiety bearing an N-hydroxysuccinimidyl (NHS) ester and allow it to react with the antibody or fusion protein under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody or fusion protein to each other may vary depending upon the particular linker, agent and/or antibody or fusion protein and functional groups selected and employed for conjugating the various components to each other.

### Methods of Producing Antibodies

Using the information provided herein, the anti-HAstV capsid spike protein antibodies and fusion proteins of the present disclosure may be prepared using standard techniques well known to those of skill in the art. For example, a nucleic acid sequence(s) encoding the amino acid sequence of an antibody or fusion protein of the present disclosure can be used to express the antibodies or fusion proteins. The polypeptide sequences provided herein (see, e.g., Table 3) can be used to determine appropriate nucleic acid sequences encoding the antibodies or fusion proteins and the nucleic acids sequences then used to express one or more antibodies or fusion proteins specific for the HAstV capsid spike protein. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject antibody is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies and fusion proteins of the present disclosure can be achieved by operably linking a nucleic acid encoding the antibody or fusion protein to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful.

Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody or fusion protein is isolated and cloned, one can express the nucleic acid in a variety of recombinantly engineered cells known to those of skill in the art. Examples of such cells include bacteria, yeast, filamentous fungi, insect (e.g., those employing baculoviral vectors), and mammalian cells.

Isolation and purification of a subject antibody or fusion protein can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies and fusion proteins of the present disclosure may contain modifications to facilitate isolation, as discussed above.

The antibodies and fusion proteins may be prepared in substantially pure or isolated form (e.g., free from other polypeptides). The protein can be present in a composition that is enriched for the polypeptide relative to other components that may be present (e.g., other polypeptides or other host cell components). Purified antibodies and fusion proteins may be provided such that the antibody or fusion protein is present in a composition that is substantially free of other expressed proteins, e.g., less than 90%, usually less than 60% and more usually less than 50% of the composition is made up of other expressed proteins.

The antibodies and fusion proteins produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies and fusion proteins of the present disclosure, where suitable cells include eukaryotic cells, e.g., mammalian cells. The cells can be a hybrid cell or “hybridoma” that is capable of reproducing antibodies in vitro (e.g., monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody or fusion protein of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia), insect or mammalian expression system, for example. One example of a suitable technique uses a bacteriophage lambda vector system having a leader sequence that causes the expressed antibody (e.g. Fab or scFv) to migrate to the periplasmic space (between the bacterial cell membrane and the cell wall) or to be secreted. One can rapidly generate great numbers of functional fragments (e.g. Fab or scFv) for those which bind the antigen of interest.

Antibodies that specifically bind a HAstV capsid spike protein can be prepared using a wide variety of techniques known in the art including the use of hybridoma, recombinant, phage display technologies, Selected Lymphocyte Antibody Method (SLAM), or a combination thereof.

For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds HAstV capsid spike protein can be selected or identified with HAstV capsid spike protein, e.g., using labeled HAstV capsid spike protein bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene Ill or gene VIII protein. The production of high affinity human antibodies by chain shuffling is known, as are combinatorial infection and in vivo recombination as a strategy for constructing large phage libraries. In another embodiment, ribosomal display can be used to replace bacteriophage as the display platform. Cell surface libraries may be screened for antibodies. Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

### Nucleic Acids, Expression Vectors and Cells

In view of the section above regarding methods of producing the antibodies and fusion proteins of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody or fusion protein of the present disclosure, including any of the anti-HAstV capsid spike protein antibodies of the present disclosure, e.g., any of such antibodies described elsewhere herein, e.g., any of the antibodies having the amino acid sequences provided in Table 3 below. According to some embodiments, the antibody is a single chain antibody (e.g., an scFv), and the nucleic acid encodes the single chain antibody.

Also provided are expression vectors comprising any of the nucleic acids of the present disclosure. Expression of natural or synthetic nucleic acids encoding the antibodies and fusion proteins of the present disclosure can be achieved by operably linking a nucleic acid encoding the antibody or fusion protein to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such as cell comprises a first expression vector comprising the first nucleic acid, and a second expression vector comprising the second nucleic acid.

Also provided are methods of making an antibody or fusion protein of the present disclosure, the methods including culturing a cell of the present disclosure under conditions suitable for the cell to express the antibody or fusion protein, wherein the antibody or fusion protein is produced. The conditions for culturing the cell such that the antibody or fusion protein is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

### Compositions

As summarized above, the present disclosure also provides compositions. According to some embodiments, a composition of the present disclosure includes an antibody, fusion protein, or conjugate of the present disclosure. For example, the antibody, fusion protein, or conjugate may be any of the antibodies, fusion proteins, or conjugates described in the Antibodies section hereinabove and/or the Experimental section below, which descriptions are incorporated but not reiterated herein for purposes of brevity.

In certain aspects, a composition of the present disclosure includes the antibody, fusion protein, or conjugate present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes an anti-HAstV capsid spike protein antibody of the present disclosure (or conjugate or fusion protein comprising same), and a pharmaceutically acceptable carrier.

The antibodies, fusion proteins, or conjugates can be incorporated into a variety of formulations for therapeutic administration. More particularly, the antibodies, fusion proteins, or conjugates can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the antibodies, fusion proteins, or conjugates for administration to an individual (e.g., suitable for human administration) are generally sterile and may further be free of detectable pyrogens or other contaminants contraindicated for administration to a patient according to a selected route of administration.

In pharmaceutical dosage forms, the antibodies, fusion proteins, or conjugates can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the antibodies, fusion proteins, or conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The antibodies, fusion proteins, or conjugates can be formulated for parenteral (e.g., intravenous, intra-arterial, intraosseous, intramuscular, intracerebral, intracerebroventricular, intrathecal, subcutaneous, etc.) administration. In certain aspects, the antibodies, fusion proteins, or conjugates are formulated for injection by dissolving, suspending or emulsifying the antibodies, fusion proteins, or conjugates in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the antibodies, fusion proteins, or conjugates may be prepared by mixing the antibodies, fusion proteins, or conjugates having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the antibodies, fusion proteins, or conjugates may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the antibody, fusion protein or conjugate against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the antibody, fusion protein, or conjugate, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure further include kits. The kits find use, e.g., in practicing the methods of the present disclosure. In some embodiments, a subject kit includes a composition (e.g., a pharmaceutical composition) that includes any of the anti-HAstV capsid spike protein antibodies, fusion proteins, conjugates, or cells of the present disclosure, e.g., any of the anti-HAstV capsid spike protein antibodies, fusion proteins, conjugates, or cells described elsewhere herein. In some embodiments, provided are kits that include any of the pharmaceutical compositions described herein, including any of the pharmaceutical compositions described above in the section relating to the compositions of the present disclosure. Kits of the present disclosure may include instructions for administering the pharmaceutical composition to an individual in need thereof, including but not limited to, an individual having or suspected of having a HAstV infection, e.g., an individual having astroviral gastroenteritis, astroviral encephalitis, and/or the like.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes any of the anti-HAstV capsid spike protein antibodies, fusion proteins, conjugates, or cells of the present disclosure (e.g., any of the anti-HAstV capsid spike protein antibodies, fusion proteins, conjugates, or cells described elsewhere herein, including those presented in Table 3). The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular anti-HAstV capsid spike protein antibody employed, the effect to be achieved, and the pharmacodynamics associated with the anti-HAstV capsid spike protein antibody, in the individual. In yet other embodiments, the kits may include a single multi dosage amount of the composition.

As will be appreciated, the kits of the present disclosure may include any of the agents and features described in the sections herein relating to the subject antibodies, methods and compositions, which are not reiterated in detail herein for purposes of brevity.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods

Aspects of the present disclosure include methods comprising administering to an individual in need thereof (e.g., an individual having a HAstV infection) an anti-HAstV capsid spike protein antibody of the present disclosure (or conjugate or fusion protein comprising same). In certain embodiments, provided are methods of treating an individual having or suspected of having a HAstV infection, the method comprising administering to the individual a therapeutically effective amount of any of the antibodies, fusion proteins, or conjugates of the present disclosure.

The anti-HAstV capsid spike protein antibodies, fusion proteins, or conjugates may be administered to any of a variety of individuals. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a HAstV, e.g., an animal model of astroviral gastroenteritis and/or astroviral encephalitis.

The anti-HAstV capsid spike protein antibodies, fusion proteins, or conjugates are administered in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a HAstV infection (e.g., a symptom of astroviral gastroenteritis and/or astroviral encephalitis), as compared to a control. In some embodiments, the therapeutically effective amount is sufficient to slow the progression of, or reduce, one or more symptoms of a HAstV infection (e.g., one or more astroviral gastroenteritis and/or astroviral encephalitis symptoms), including but not limited to, viral load, diarrhea, vomiting, nausea, anxiety, headache, malaise, abdominal discomfort, fever, and/or the like. According to some embodiments, the therapeutically effective amount slows the progression of, or reduces, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the anti-HAstV capsid spike protein antibodies, fusion proteins, conjugates or cells. An effective amount can be administered in one or more administrations.

When the methods include administering a combination of the anti-HAstV capsid spike protein antibody (or fusion protein or conjugate) and a second agent (e.g., a second anti-HAstV capsid spike protein antibody (or fusion protein or conjugate) of the present disclosure; or a second agent approved for treatment of a HAstV infection, the anti-HAstV capsid spike protein antibody and the second agent may be administered concurrently (e.g., in the same or separate formulations), sequentially, or both. For example, according to certain embodiments, the second agent is administered to the individual prior to administration of the anti-HAstV capsid spike protein antibody, concurrently with administration of the anti-HAstV capsid spike protein antibody, or both. In some embodiments, the anti-HAstV capsid spike protein antibody is administered to the individual prior to administration of the second agent, concurrently with administration of the second agent, or both.

In certain aspects, the one or more agents are administered according to a dosing regimen approved for individual use. In some embodiments, the administration of the anti-HAstV capsid spike protein antibody permits the second agent to be administered according to a dosing regimen that involves one or more lower and/or less frequent doses, and/or a reduced number of cycles as compared with that utilized when the second agent is administered without administration of the anti-HAstV capsid spike protein antibody. In some embodiments, the administration of the second agent permits the anti-HAstV capsid spike protein antibody to be administered according to a dosing regimen that involves one or more lower and/or less frequent doses, and/or a reduced number of cycles as compared with that utilized when the anti-HAstV capsid spike protein antibody is administered without administration of the second agent.

As noted above, in certain embodiments, one or more doses of the anti-HAstV capsid spike protein antibody and second agent are administered at the same time; in some such embodiments, such agents may be administered present in the same pharmaceutical composition. In some embodiments, however, the anti-HAstV capsid spike protein antibody and second agent are administered to the individual in different compositions and/or at different times. For example, the anti-HAstV capsid spike protein antibody may be administered prior to administration of the second agent (e.g., in a particular cycle). Alternatively, the second agent may be administered prior to administration of the anti-HAstV capsid spike protein antibody (e.g., in a particular cycle). The second agent to be administered may be administered a period of time that starts at least 1 hour, 3 hours, 6 hours, 12 hours, 24 hours, 48 hours, 72 hours, or up to 5 days or more after the administration of the first agent.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

In some embodiments, the anti-HAstV capsid spike protein antibody and second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating the effect of the anti-HAstV capsid spike protein antibody), may include: (a) a first dosing period during which a therapeutically effective amount a first agent is administered to an individual; (b) a first resting period; (c) a second dosing period during which a therapeutically effective amount of a second agent and, optionally, a third agent, is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include the identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The anti-HAstV capsid spike protein antibody, and if also administered, a second agent, may be administered via a route of administration independently selected from oral, parenteral (e.g., by intravenous, intra-arterial, subcutaneous, intramuscular, or epidural injection), topical, or nasal administration. According to certain embodiments, the anti-HAstV capsid spike protein antibody is administered parenterally.

As described above, aspects of the present disclosure include methods for treating an individual having or suspected of having a HAstV infection, e.g., COVID-19. By treatment is meant at least an amelioration of one or more symptoms associated with the HAstV infection (e.g., astroviral gastroenteritis and/or astroviral encephalitis) of the individual, where amelioration is used in a broad sense to refer to at least a reduction in the magnitude of a parameter, e.g., symptom, associated with the HAstV infection. Non-limiting examples of such symptoms include one or more of viral load, diarrhea, vomiting, nausea, anxiety, headache, malaise, abdominal discomfort, fever, and/or the like. As such, treatment also includes situations where the HAstV infection, or at least one or more symptoms associated therewith, are completely inhibited, e.g., prevented from happening, or stopped, e.g., terminated, such that the individual no longer suffers from the HAstV infection, or at least the symptoms that characterize the HAstV infection.

Notwithstanding the appended claims, the present disclosure is also defined by the following embodiments:

1. An antibody that specifically binds to a human astrovirus (HAstV) capsid spike protein and competes for binding to the HAstV capsid spike protein with an antibody comprising:
a variable heavy chain (V_{H}) polypeptide comprising
   a V_{H} CDR1 comprising the amino acid sequence GFSLTSYGVH (SEQ ID NO:2),
   a V_{H} CDR2 comprising the amino acid sequence VIWSGGSTDYNAAFIS (SEQ ID NO:3), and
   a V_{H} CDR3 comprising the amino acid sequence NSLLDAMDY (SEQ ID NO:4), and
a variable light chain (V_{L}) polypeptide comprising
   a V_{L} CDR1 comprising the amino acid sequence KASQSVSNAVA (SEQ ID NO:6),
   a V_{L} CDR2 comprising the amino acid sequence YASNRYT (SEQ ID NO:7), and
   a V_{L} CDR3 comprising the amino acid sequence QQDYSSPLT (SEQ ID NO:8);
a variable heavy chain (V_{H}) polypeptide comprising
   a V_{H} CDR1 comprising the amino acid sequence GYTFTSYWMH (SEQ ID NO:10),
   a V_{H} CDR2 comprising the amino acid sequence EINPSSGRANYNEKFKN (SEQ ID NO:11), and
   a V_{H} CDR3 comprising the amino acid sequence DYYAMDY (SEQ ID NO:12), and
a variable light chain (V_{L}) polypeptide comprising
   a VL CDR1 comprising the amino acid sequence RASQSISNNLH (SEQ ID NO:14),
   a V_{L} CDR2 comprising the amino acid sequence SASQSIS (SEQ ID NO:15), and
   a V_{L} CDR3 comprising the amino acid sequence QQTNSWPLT (SEQ ID NO:16);
a variable heavy chain (V_{H}) polypeptide comprising
   a V_{H} CDR1 comprising the amino acid sequence GYSITSGYYWN (SEQ ID NO:18),
   a V_{H} CDR2 comprising the amino acid sequence YISYDGSNNYNPSLKN (SEQ ID NO:19), and
   a V_{H} CDR3 comprising the amino acid sequence FYDGYDY (SEQ ID NO:20), and
a variable light chain (V_{L}) polypeptide comprising
   a V_{L} CDR1 comprising the amino acid sequence KASQDVSTAVA (SEQ ID NO:22),
   a V_{L} CDR2 comprising the amino acid sequence WASTRHT (SEQ ID NO:23), and
   a V_{L} CDR3 comprising the amino acid sequence QQHYSTPFT (SEQ ID NO:24);
a variable heavy chain (V_{H}) polypeptide comprising
   a V_{H} CDR1 comprising the amino acid sequence GFSLTTFGIH (SEQ ID NO:26),
   a V_{H} CDR2 comprising the amino acid sequence VIWAAGTTNYNSTLKS (SEQ ID NO:27), and
   a VH CDR3 comprising the amino acid sequence EDYDYFFGLDY (SEQ ID NO:28), and
a variable light chain (V_{L}) polypeptide comprising
   a V_{L} CDR1 comprising the amino acid sequence RSSTGAVTTSNYAS (SEQ ID NO:30),
   a V_{L} CDR2 comprising the amino acid sequence GTNNRAP (SEQ ID NO:31), and
   a VL CDR3 comprising the amino acid sequence ALWFSNHWV (SEQ ID NO:32);
a variable heavy chain (V_{H}) polypeptide comprising
   a V_{H} CDR1 comprising the amino acid sequence GFSLTSYGVH (SEQ ID NO:1),
   a V_{H} CDR2 comprising the amino acid sequence VIWADGSTNYNSALMS (SEQ ID NO:34), and
   a V_{H} CDR3 comprising the amino acid sequence WTYGDYFDY (SEQ ID NO:35), and
a variable light chain (V_{L}) polypeptide comprising
   a V_{L} CDR1 comprising the amino acid sequence SASQGISNYLN (SEQ ID NO:37),
   a V_{L} CDR2 comprising the amino acid sequence YTSSLHS (SEQ ID NO:38), and
   a V_{L} CDR3 comprising the amino acid sequence QQYSKLPYT (SEQ ID NO:39);
a variable heavy chain (V_{H}) polypeptide comprising
   a VH CDR1 comprising the amino acid sequence GFTFSYFGMH (SEQ ID NO:41),
   a V_{H} CDR2 comprising the amino acid sequence YISSGSNTIYYADTVKG (SEQ ID NO:42), and
   a V_{H} CDR3 comprising the amino acid sequence AYYGNHYYAMDF (SEQ ID NO:43), and
a variable light chain (V_{L}) polypeptide comprising
   a V_{L} CDR1 comprising the amino acid sequence RASENIYSNLA (SEQ ID NO:45),
   a V_{L} CDR2 comprising the amino acid sequence AARDLAT (SEQ ID NO:46), and
   a V_{L} CDR3 comprising the amino acid sequence QHFWETPWT (SEQ ID NO:47); or
a variable heavy chain (V_{H}) polypeptide comprising
   a V_{H} CDR1 comprising the amino acid sequence GFTFSDYYMY (SEQ ID NO:49),
   a V_{H} CDR2 comprising the amino acid sequence TISDGGFSTYYPDSVKG (SEQ ID NO:50), and
   a V_{H} CDR3 comprising the amino acid sequence FGAYSTF (SEQ ID NO:51), and
a variable light chain (V_{L}) polypeptide comprising
   a V_{L} CDR1 comprising the amino acid sequence RASSSVSYMY (SEQ ID NO:53),
   a V_{L} CDR2 comprising the amino acid sequence ATSNLAS (SEQ ID NO:54), and
   a V_{L} CDR3 comprising the amino acid sequence QQWSSDPPMT (SEQ ID NO:55).
      2. The antibody of embodiment 1, wherein the antibody comprises:
a variable heavy chain (V_{H}) polypeptide comprising
   a V_{H} CDR1 comprising the amino acid sequence GFSLTSYGVH (SEQ ID NO:2),
   a V_{H} CDR2 comprising the amino acid sequence VIWSGGSTDYNAAFIS (SEQ ID NO:3), and
   a VH CDR3 comprising the amino acid sequence NSLLDAMDY (SEQ ID NO:4), and
a variable light chain (V_{L}) polypeptide comprising
   a V_{L} CDR1 comprising the amino acid sequence KASQSVSNAVA (SEQ ID NO:6),
   a V_{L} CDR2 comprising the amino acid sequence YASNRYT (SEQ ID NO:7), and
   a V_{L} CDR3 comprising the amino acid sequence QQDYSSPLT (SEQ ID NO:8);
a variable heavy chain (V_{H}) polypeptide comprising
   a V_{H} CDR1 comprising the amino acid sequence GYTFTSYWMH (SEQ ID NO:10),
   a V_{H} CDR2 comprising the amino acid sequence EINPSSGRANYNEKFKN (SEQ ID NO:11), and
   a V_{H} CDR3 comprising the amino acid sequence DYYAMDY (SEQ ID NO:12), and
a variable light chain (V_{L}) polypeptide comprising
   a V_{L} CDR1 comprising the amino acid sequence RASQSISNNLH (SEQ ID NO:14),
   a V_{L} CDR2 comprising the amino acid sequence SASQSIS (SEQ ID NO:15), and
   a V_{L} CDR3 comprising the amino acid sequence QQTNSWPLT (SEQ ID NO:16);
a variable heavy chain (V_{H}) polypeptide comprising
   a V_{H} CDR1 comprising the amino acid sequence GYSITSGYYWN (SEQ ID NO:18),
   a VH CDR2 comprising the amino acid sequence YISYDGSNNYNPSLKN (SEQ ID NO:19), and
   a V_{H} CDR3 comprising the amino acid sequence FYDGYDY (SEQ ID NO:20), and
a variable light chain (V_{L}) polypeptide comprising
   a V_{L} CDR1 comprising the amino acid sequence KASQDVSTAVA (SEQ ID NO:22),
   a V_{L} CDR2 comprising the amino acid sequence WASTRHT (SEQ ID NO:23), and
   a V_{L} CDR3 comprising the amino acid sequence QQHYSTPFT (SEQ ID NO:24);
a variable heavy chain (V_{H}) polypeptide comprising
   a V_{H} CDR1 comprising the amino acid sequence GFSLTTFGIH (SEQ ID NO:26),
   a V_{H} CDR2 comprising the amino acid sequence VIWAAGTTNYNSTLKS (SEQ ID NO:27), and
   a V_{H} CDR3 comprising the amino acid sequence EDYDYFFGLDY (SEQ ID NO:28), and
a variable light chain (V_{L}) polypeptide comprising
   a V_{L} CDR1 comprising the amino acid sequence RSSTGAVTTSNYAS (SEQ ID NO:30),
   a V_{L} CDR2 comprising the amino acid sequence GTNNRAP (SEQ ID NO:31), and
   a V_{L} CDR3 comprising the amino acid sequence ALWFSNHWV (SEQ ID NO:32);
a variable heavy chain (V_{H}) polypeptide comprising
   a V_{H} CDR1 comprising the amino acid sequence GFSLTSYGVH (SEQ ID NO:1),
   a V_{H} CDR2 comprising the amino acid sequence VIWADGSTNYNSALMS (SEQ ID NO:34), and
   a V_{H} CDR3 comprising the amino acid sequence WTYGDYFDY (SEQ ID NO:35), and
a variable light chain (V_{L}) polypeptide comprising
   a V_{L} CDR1 comprising the amino acid sequence SASQGISNYLN (SEQ ID NO:37),
   a V_{L} CDR2 comprising the amino acid sequence YTSSLHS (SEQ ID NO:38), and
   a V_{L} CDR3 comprising the amino acid sequence QQYSKLPYT (SEQ ID NO:39);
a variable heavy chain (V_{H}) polypeptide comprising
   a V_{H} CDR1 comprising the amino acid sequence GFTFSYFGMH (SEQ ID NO:41),
   a V_{H} CDR2 comprising the amino acid sequence YISSGSNTIYYADTVKG (SEQ ID NO:42), and
   a VH CDR3 comprising the amino acid sequence AYYGNHYYAMDF (SEQ ID NO:43), and
a variable light chain (V_{L}) polypeptide comprising
   a V_{L} CDR1 comprising the amino acid sequence RASENIYSNLA (SEQ ID NO:45),
   a V_{L} CDR2 comprising the amino acid sequence AARDLAT (SEQ ID NO:46), and
   a V_{L} CDR3 comprising the amino acid sequence QHFWETPWT (SEQ ID NO:47); or a variable heavy chain (V_{H}) polypeptide comprising
   a V_{H} CDR1 comprising the amino acid sequence GFTFSDYYMY (SEQ ID NO:49),
   a V_{H} CDR2 comprising the amino acid sequence TISDGGFSTYYPDSVKG (SEQ ID NO:50), and
   a V_{H} CDR3 comprising the amino acid sequence FGAYSTF (SEQ ID NO:51), and
a variable light chain (V_{L}) polypeptide comprising
   a V_{L} CDR1 comprising the amino acid sequence RASSSVSYMY (SEQ ID NO:53),
   a V_{L} CDR2 comprising the amino acid sequence ATSNLAS (SEQ ID NO:54), and
   a V_{L} CDR3 comprising the amino acid sequence QQWSSDPPMT (SEQ ID NO:55).
      3. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:1; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:5.
   4. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:9; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:13.
   5. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:21.
   6. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:25; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:29.
   7. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:33; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:36.
   8. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:40; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:44.
   9. The antibody of embodiment 1 or embodiment 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:48; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:52.
   10. The antibody of any one of embodiments 1 to 9, wherein the antibody is a monoclonal antibody.
   11. The antibody of any one of embodiments 1 to 10, wherein the antibody is a humanized antibody.
   12. The antibody of any one of embodiments 1 to 11, wherein the antibody is an IgG.
   13. The antibody of embodiment 12, wherein the antibody comprises a human Fc domain.
   14. The antibody of embodiment 13, wherein the human Fc domain is a human IgG1 Fc domain.
   15. The antibody of any one of embodiments 1 to 11, wherein the antibody is selected from the group consisting of: a Fab, a F(ab′)₂, and a F(ab′).
   16. The antibody of any one of embodiments 1 to 11, wherein the antibody is a single chain antibody.
   17. The antibody of embodiment 16, wherein the single chain antibody is an scFv.
   18. The antibody of any one of embodiments 1 to 17, wherein the antibody is a bispecific antibody comprising a first antigen-binding domain comprising a V_{H} polypeptide-V_{L} polypeptide pair as defined in any one of embodiments 1 to 9.
   19. The antibody of embodiment 18, wherein the bispecific antibody comprises a second antigen-binding domain that specifically binds a different epitope on the HAstV capsid spike protein as compared to the epitope specifically bound by the first antigen-binding domain.
   20. The antibody of embodiment 18, wherein the bispecific antibody comprises a second antigen-binding domain that specifically binds an antigen other than a HAstV capsid spike protein.
   21. A fusion protein, comprising:
a chain of an antibody of any one of embodiments 1 to 20 fused to a heterologous sequence of amino acids.
   22. The fusion protein of embodiment 21, wherein the heterologous sequence of amino acids is fused to the C-terminus of the chain of the antibody.
   23. The fusion protein of embodiment 21 or embodiment 22, wherein the antibody is the single chain antibody of embodiment 16 or 17.
   24. A conjugate, comprising:
an antibody of any one of embodiments 1 to 20 or a fusion protein of any one of embodiments 21 to 23; and
an agent conjugated to the antibody or fusion protein.
   25. The conjugate of embodiment 24, wherein the agent is a detectable label or a half-life extending moiety.
   26. The conjugate of embodiment 24 or embodiment 25, wherein the agent is conjugated to the antibody via a non-cleavable linker.
   27. The conjugate of embodiment 24 or embodiment 25, wherein the agent is conjugated to the antibody via a cleavable linker.
   28. A nucleic acid encoding a variable heavy chain (V_{H}) polypeptide, a variable light chain (V_{L}) polypeptide, or both, of an antibody of any one of embodiments 1 to 20.
   29. A nucleic acid encoding the fusion protein of any one of embodiments 21 to 23.
   30. An expression vector comprising the nucleic acid of embodiment 28 or embodiment 29.
   31. A cell comprising:
the nucleic acid of embodiment 28 or embodiment 29; or
the expression vector of embodiment 30.
   32. A cell comprising:
a first nucleic acid encoding the variable heavy chain (V_{H}) polypeptide of an antibody of any one of embodiments 1 to 15; and
a second nucleic acid encoding the variable light chain (V_{L}) polypeptide of the antibody.
   33. The cell of embodiment 32, comprising:
a first expression vector comprising the first nucleic acid; and
a second expression vector comprising the second nucleic acid.
   34. A method of producing the antibody of any one of embodiments 1 to 15, comprising culturing the cell of any one of embodiments 31 to 33 under conditions suitable for the cell to express the antibody, wherein the antibody is produced.
   35. A pharmaceutical composition, comprising:
the antibody of any one of embodiments 1 to 20; and
a pharmaceutically acceptable carrier.
   36. A pharmaceutical composition, comprising:
the fusion protein of any one of embodiments 21 to 23; and
a pharmaceutically acceptable carrier.
   37. A pharmaceutical composition, comprising:
the conjugate of any one of embodiments 24 to 27; and
a pharmaceutically acceptable carrier.
   38. A kit, comprising:
the pharmaceutical composition of any one of embodiments 35 to 37; and
instructions for administering the pharmaceutical composition to an individual in need thereof.
   39. The kit of embodiment 38, wherein the pharmaceutical composition is present in one or more unit dosages.
   40. The kit of embodiment 38, wherein the pharmaceutical composition is present in two or more unit dosages.
   41. A method, comprising:
administering to an individual having a human astrovirus (HAstV) infection the antibody of any one of embodiments 1 to 20, the fusion protein of any one of embodiments 21 to 23, or the conjugate of any one of embodiments 24 to 27.
   42. A method of treating an individual having a human astrovirus (HAstV) infection, the method comprising:
administering to the individual a therapeutically effective amount of the antibody of any one of embodiments 1 to 20, the fusion protein of any one of embodiments 21 to 23, or the conjugate of any one of embodiments 24 to 27.
   43. The method according to embodiment 41 or embodiment 42, wherein the infection is a HAstV serotype 1 (HAstV1) infection, a HAstV serotype 2 (HAstV2) infection, a HAstV serotype 3 (HAstV3) infection, a HAstV serotype 4 (HAstV4) infection, a HAstV serotype 5 (HAstV5) infection, a HAstV serotype 6 (HAstV6) infection, a HAstV serotype 7 (HAstV7) infection, or a HAstV serotype 1 (HAstV8) infection.
   44. The method according to embodiment 41 or embodiment 42, wherein the infection is a HAstV serotype 1 (HAstV1) infection.
   45. The method according to any one of embodiments 41 to 44, wherein the individual has astroviral gastroenteritis.
   46. The method according to any one of embodiments 41 to 45, wherein the individual has astroviral encephalitis.

The following examples are offered by way of illustration and not by way of limitation.

### EXPERIMENTAL

### Example 1—Neutralizing Antibodies 3E8 and 2D9 have High Affinity for the HAstV8 Capsid Spike

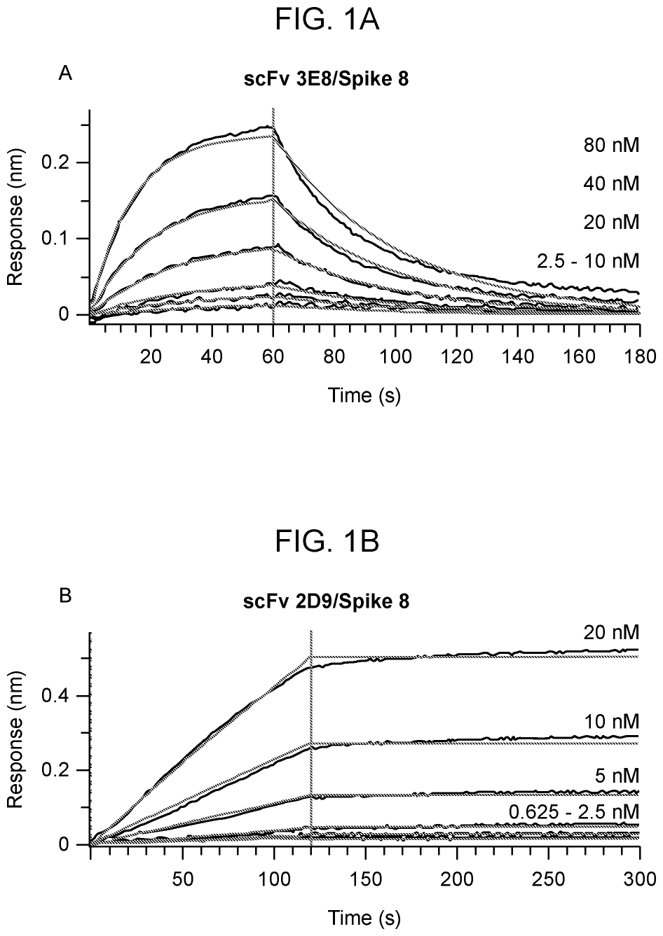

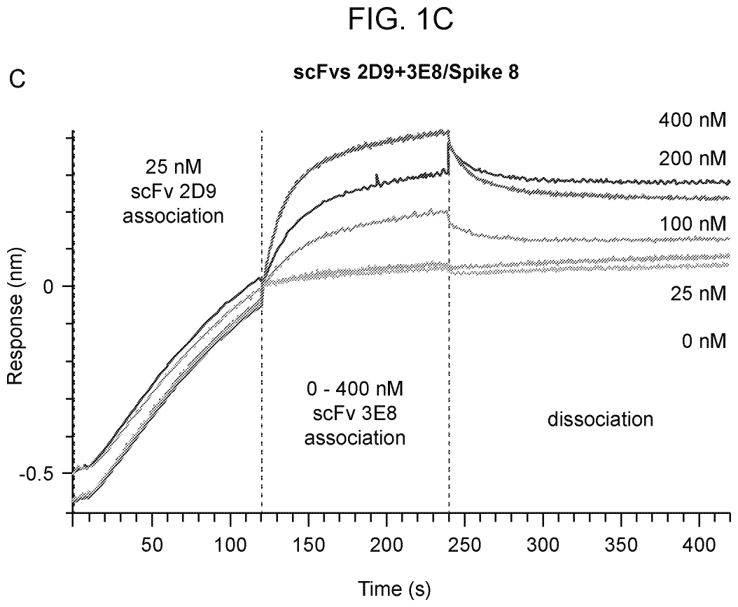

Previously, mouse monoclonal antibodies (mAbs) 3E8 and 2D9 were raised against recombinant Spike 8 in mice and shown to neutralize infectivity of HAstV8 in human colon adenocarcinoma cells (Caco-2 cells), the standard cell line for HAstV propagation.⁴¹ In the present study, the affinities of purified recombinant antibodies 3E8 and 2D9 for purified recombinant Spike 8 were first examined. To assess and quantify binding, biolayer interferometry (BLI) experiments were performed. Single-chain variable fragment (scFv) antibody constructs were used for these kinetic studies to eliminate potential complications of avidity with bivalent antibody and dimeric Spike 8. After a baseline measurement, Spike 8 fused to a 10× histidine tag was loaded onto Anti-Penta-His biosensors, which were subsequently submerged in 1:2 serial dilutions of scFv to measure on-rates, followed by submersion in buffer to measure off-rates. scFv 3E8 and scFv 2D9 were found to bind to Spike 8 in a dose-dependent manner with high-affinity dissociation constants (K_{D}) of 40.03±2.37 nM and 2.45±0.26 pM, respectively (FIG. 1, panels A and B, and Table 1). It was concluded from these results that immunization of mice with the Spike 8 antigen produces high-affinity HAstV8-neutralizing antibodies. These studies support the development of a subunit vaccine for HAstV using recombinant spikes as immunogens.

TABLE 1
scFv 3E8 and scFv 2D9 bind Spike 8 with high affinity

| | K_{D} ± σ | R² | χ² |
|---|---|---|---|
| scFv 3E8/Spike 8 | 40.03 ± 2.37 nM | 0.9900 | 0.4435 |
| scFv 2D9/Spike 8 | 2.45 ± 0.26 pM | 0.9968 | 0.8963 |

Next, a competition BLI experiment was performed to determine whether 3E8 and 2D9 compete for the same target on Spike 8 or whether they can bind Spike 8 simultaneously. Spike 8 was loaded onto the biosensors and scFv 2D9, which has the stronger affinity, was first allowed to bind. Association of the higher-affinity scFv 2D9 first ensured that it would not be displaced upon association of the weaker-affinity scFv 3E8. The biosensors were then dipped into dilutions of scFv 3E8 and an additional association signal was observed, suggesting that 3E8 and 2D9 can bind Spike 8 at the same time and therefore at different epitopes (FIG. 1C).

This result is in accordance with escape mutation studies done previously, in which HAstV8 escape mutants were generated using mouse mAbs 3E8 and 2D9.⁴¹ Virus was cultured with low concentrations of one mouse mAb. Under selective pressure, the virus mutated to escape mAb neutralization. After sequencing, the viral variant able to escape neutralization by mouse mAb 3E8 contained the mutation Y464H in the Spike 8 sequence. In contrast, the viral variant that can escape neutralization by mouse mAb 2D9 contained the mutation D597Y in the Spike 8 sequence. Both of these mutations map to the surface of Spike 8, but are ˜30 A distant from each other in space, hinting at non-overlapping epitopes. In addition, mAb 3E8 still effectively neutralized the mAb 2D9 viral escape mutant and vice versa. These results suggest that the Spike 8 epitopes recognized by mAb 3E8 and mAb 2D9 are situated in different locations on the Spike 8 surface but do not provide a complete picture of the epitope footprint(s) present on Spike 8.

### Example 2 - scFv 3E8 Binds Spike 8 at a Known Epitope

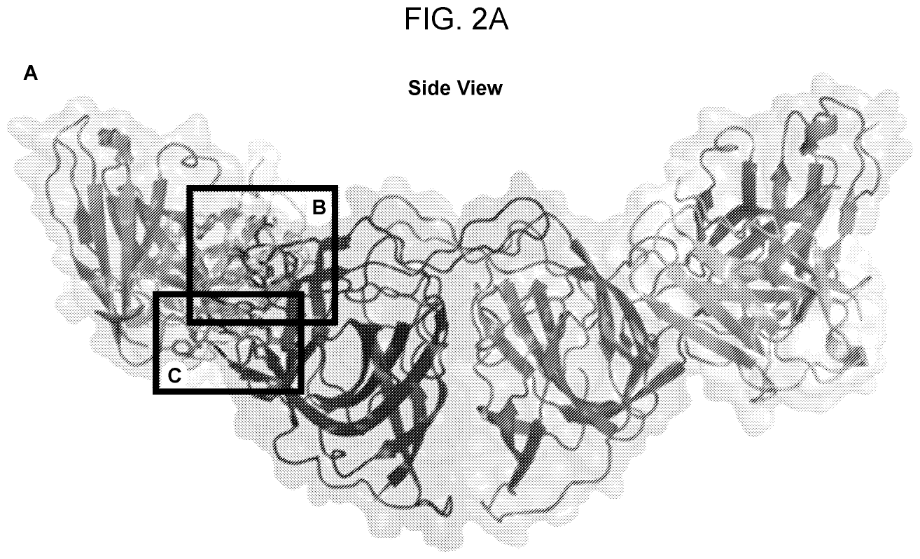

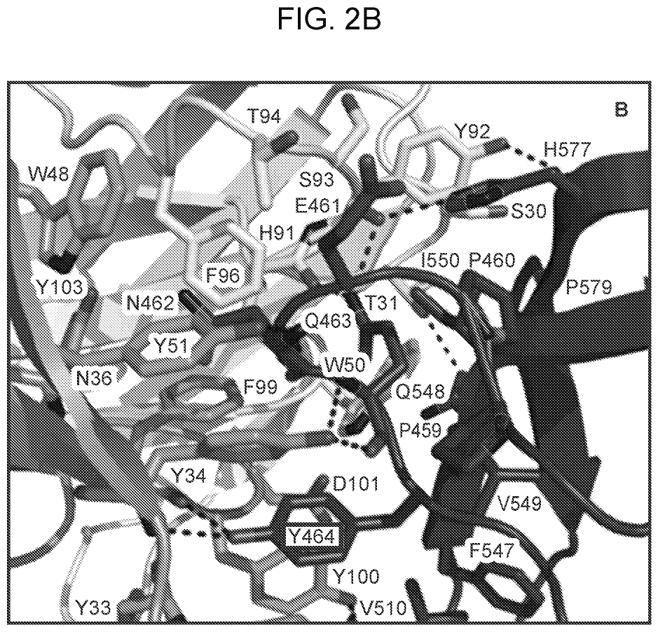

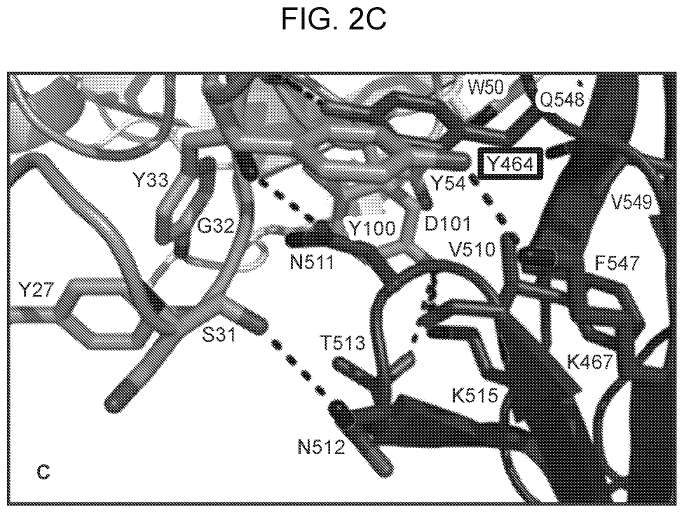

Previously, the structure of a HAstV2-specific neutralizing antibody, PL-2, in complex with the HAstV2 capsid spike (Spike 2) was solved.⁴⁰ The PL-2 antibody mainly contacts Spike 2 Loop 1, and the epitope contains residues directly adjacent to 3E8 escape mutant Y464. Therefore, PL-2 and 3E8 may target a similar epitope. To define the mAb 3E8 binding site on Spike 8, scFv 3E8 and Spike 8 were co-crystallized and the structure of the scFv 3E8/Spike 8 complex were solved to 2.05 Å resolution (FIG. 2 and Table 2). The structure confirms size exclusion chromatography data demonstrating that scFv 3E8 binds to Spike 8 in a 2:2 ratio, whereby one scFv 3E8 molecule makes contact with one protomer of the Spike 8 dimer, and a second scFv 3E8 molecule binds the same epitope on the other Spike 8 protomer (FIG. 2). A conformation-dependent tertiary epitope on each Spike 8 protomer that overlaps with the PL-2 epitope was observed (discussed further below). Four segments of amino acids from one linear Spike 8 molecule comprise the three-dimensional 3E8-targeted epitope.

TABLE 2
Data collection and refinement statistics
for scFv 3E8/Spike8 and scFv 2D9/Spike

| Characteristic | scFv 3E8/Spike 8 | scFv 2D9/Spike 8 |
|---|---|---|
| PDB code | 7RK1 | 7RK2 |
| Data collection | | |
| Space group | P 1 21 1 | P 21 21 21 |
| Cell dimensions | | |
| a, b, c (Å) | 48.83, 80.43, 117.21 | 49.59, 97.34, 208.31 |
| α, β, γ (°) | 90.00, 90.67, 90.00 | 90.00, 90.00, 90.00 |
| Resolution (Å) | 80.43-2.05 (2.11-2.05) | 208.31-2.65 (2.78-2.65) |
| R_{sym} or R_{merge} | 0.184 (1.268) | 0.451 (3.877) |
| I/σI | 7.2 (2.2) | 16.0 (2.0) |
| Completeness (%) | 97.9 (94.8) | 100 (99.8) |
| Redundancy | 6.1 (5.3) | 76.5 (66.6) |
| CC_{1/2} | 0.992 (0.602) | 0.998 (0.692) |
| Refinement | | |
| Number of reflections | 55,630 | 30,211 |
| Resolution (Å) | 66.32-2.05 | 104.15-2.65 |
| R_{work}/R_{free} | 0.223/0.238 | 0.218/0.247 |
| Number of atoms | 7378 | 7105 |
| Protein | 7043 | 6955 |
| Ligands | 0 | 0 |
| Water | 335 | 150 |
| B factors | 32.18 | 40.05 |
| Protein | 32.13 | 40.15 |
| Ligands | N/A | N/A |
| Water | 33.32 | 35.52 |
| RMSD | | |
| Bond lengths (Å) | 0.012 | 0.013 |
| Bond angles (°) | 1.70 | 1.49 |
| Ramachandran statistics | | |
| Favored (%) | 97.5 | 97.5 |
| Allowed (%) | 2.5 | 2.5 |
| Outliers (%) | 0 | 0 |

Data from one crystal were used for each structure determination. Values in parentheses are for the highest-resolution shell.

All six complementarity-determining regions (CDRs) from the kappa (κ) and heavy (H) chains of each scFv 3E8 interact with one protomer of Spike 8 (FIG. 2). The interface is defined by several contact points: (1) scFv 3E8 CDR H2 contacts Spike 8 Loop 1 residues P459, P460, E461, N462, and K467, while scFv 3E8 CDR H1 contacts N462, Q463, and Y464. CDRs K3 and H3 also contact Spike 8 Q463 and Y464, respectively (FIG. 2B). (2) scFv 3E8 CDR H3 contacts Spike 8 β5-β6 residues V510, N511, T513, and K515, and CDR H1 contacts N511 and N512 (FIG. 2C). (3) scFv 3E8 CDR H3 contacts Spike 8 β8 residue F547, CDRs K1 and K2 contact Q548, and CDRs K2 and K3 contact V549 and 1550 (FIG. 2B). (4) scFv 3E8 CDR K3 contacts Spike 8 β9 residue H577, and CDRs K1 and K3 contact P579 (FIG. 2B). scFv 3E8 binds Spike 8 through a large network of hydrogen bonds, electrostatic bonds, and van der Waals interactions. Many of the amino acids in the 3E8 epitope are unique to serotype 8, consistent with virus neutralization data demonstrating that 3E8 is serotype specific.⁴¹ However, three residues, K515, F547, and V549, are strictly conserved across all eight serotypes, while P460 and K467 are mostly conserved. Spike 8 residue Y464, the location of the Y464H escape mutant, is buried by scFv 3E8 binding (FIGS. 2B and 2C). Compared to the wild-type tyrosine, the side chain of the histidine mutant may be too short to form hydrogen bonds with the backbone nitrogen of scFv 3E8 CDR H1 residue Y34 and/or the backbone carbonyl oxygen of scFv 3E8 CDR H3 residue F99, thereby affecting antibody binding. Moreover, Y464 makes pi-stacking interactions with scFv 3E8 CDR H1, H2, and H3 residues Y34, Y54, F99, and Y100, all within 3 to 5 A, and mutation to histidine may diminish these interactions. The finding that Spike 8 residue Y464 is part of the epitope signifies that the escape mutation studies accurately pointed to the approximate location of the epitope. However, only the structure of the entire complex revealed the full extent of the scFv 3E8/Spike 8 binding interface.

### Example 3 - scFv 2D9 Binds Spike 8 at a Novel Epitope

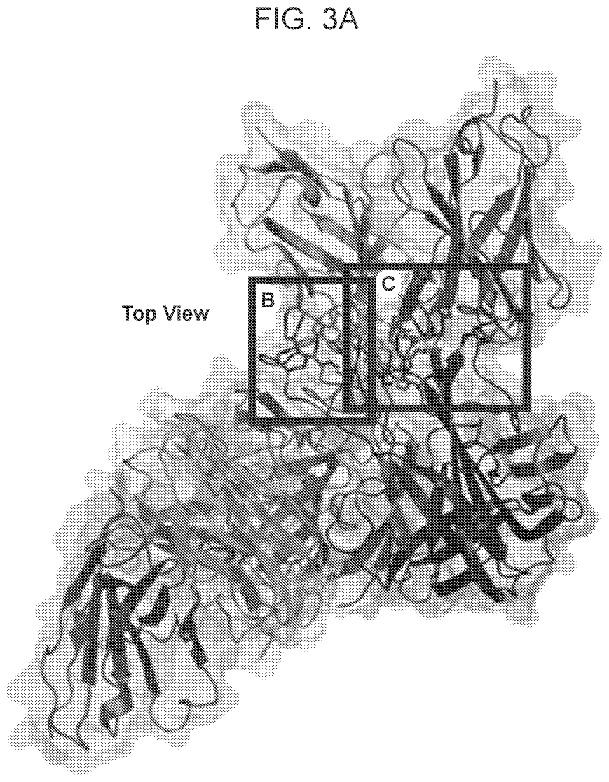

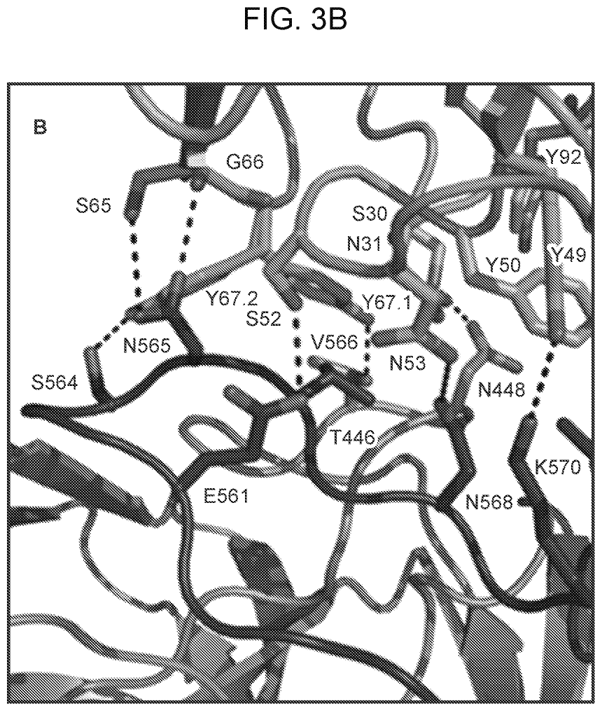

To fully characterize the mAb 2D9 binding site on Spike 8 and compare it to the 3E8 and PL-2 binding site, scFv 2D9 and Spike 8 were co-crystallized and the structure of the scFv 2D9/Spike 8 complex was solved to 2.65-A resolution (FIG. 3 and Table 2). The structure confirms size exclusion chromatography data demonstrating that scFv 2D9 also binds to Spike 8 in a 2:2 ratio, the same ratio as for scFv 3E8/Spike 8 and scFv PL-2/Spike 2. In contrast to the 3E8 tertiary epitope, it was observed that scFv 2D9 binds to a conformation-dependent quaternary epitope across the Spike 8 dimer interface that is fully distinct from the epitope targeted by 3E8 and PL-2. Five patches of amino acids from two linear Spike 8 molecules come together in the three-dimensional structure.

Five complementarity-determining regions (CDRs) from the kappa (K) and heavy (H) chains of each scFv 2D9 contact Spike 8 (FIG. 3). The interface is characterized by several interactions: (1) scFv 2D9 kappa chain residue Y67 near CDR K1, which exhibits electron density for two conformations in the structure, contacts T446 with Orientation 1 (Y67.1) in Loop 1 of one Spike 8 protomer. In addition, scFv 2D9 CDR K1 contacts N448 on that protomer (FIG. 3B). The remaining interactions take place on the other Spike 8 protomer, indicating a quaternary epitope: (2) scFv 2D9 CDR K3 contacts Spike 8 residues H528, T529, N530, and N531 in the β6-β7 junction. CDR H3 also contacts T529 and R532. CDR K2 contacts R532 (FIG. 3C). (3) scFv 2D9 CDR K2 contacts Spike 8 E561, scFv 2D9 kappa chain Y67 Orientation 2 (Y67.2) contacts Spike 8 S564, and scFv 2D9 kappa chain S65 and G66 contact N565. scFv 2D9 kappa chain Y67.1 and CDR K1 bury Spike 8 V566. CDR K2 contacts Spike 8 N568 and K570. These Spike 8 residues are all in Loop 3 (FIG. 3B). (4) scFv 2D9 CDR H3 contacts Spike 8 M595 in the β10 strand. scFv 2D9 CDR H2 contacts Spike 8 β10 residues D597, G598, and P599 (FIG. 3C). (5) scFv 2D9 CDR K2 contacts Spike 8 Loop 4 residues N614 and A616. CDR H3 buries Spike 8 1615 (FIG. 3C). Similarly to scFv 3E8, scFv 2D9 relies on a large system of hydrogen bonds, electrostatic bonds, and van der Waals interactions to bind to a quaternary epitope on the surface of the Spike 8 dimer. Notably, Spike 8 residue D597, which mutated to Y during escape mutant studies, is buried by scFv 2D9 binding (FIG. 3C). The mutation of aspartate to the larger tyrosine likely caused steric clashing with scFv 2D9 CDR H2 residue S53, completely prohibiting antibody binding.

While some amino acids in the epitope bound by scFv 2D9 are unique to HAstV8, the epitope also overlaps or abuts several patches of amino acids that are highly conserved between serotypes 1-8, suggesting that scFv 2D9 may block an important functional site on the Spike 8 surface. Interestingly, the epitope includes all four amino acids (T529, N530, N531, and R532) of an exposed, flexible β-turn knob which is very well-conserved among all serotypes and hypothesized to interact with other viral or host proteins (FIG. 3C and FIG. 4C).39 Critically, there is no overlap in the Spike 8 residues targeted by scFv 3E8 and scFv 2D9, indicating two separate and distinct epitopes on the human astrovirus capsid spike surface (FIGS. 4A and 4B).

### Example 4—Antibodies 3E8, 2D9, and PL-2 Target Epitopes Near the P Site

The human astrovirus host cell receptor is unidentified. In addition, the receptor-binding site(s) on the HAstV capsid surface is unknown. Because 3E8, 2D9, and PL-2 all inhibit virus attachment to cells,⁴⁰ they likely each prevent virus access to the receptor. Several putative receptor-binding sites on the spike surface have been proposed.³⁹ Among them is the conserved P site, located in a shallow groove on top of the spike domain. The P site has many hydrophilic residues with side chains exposed to solvent, making it highly accessible to potential cell receptors without steric hindrance. While Spike 8 residues in the 3E8, 2D9, and PL-2 epitopes mostly do not overlap with residues in the P site, the residues in the epitopes appear to frame the P sites on each Spike 8 protomer (FIG. 4B). If the P site plays a role in virus attachment to the host cell receptor, its placement between the epitopes may lead to a loss of virus access to the receptor when any of these three antibodies bind.

FIG. 4C presents a sequence alignment of the spikes from all eight classical HAstV serotypes, with the residues in the P site as well as in the epitopes targeted by 3E8, 2D9, and PL-2 indicated. Eight residues are located in the epitopes targeted by both 3E8 and PL-2 (although N511 is the only one with sequence identity between Spike 2 and Spike 8), which reveals the particular vulnerability to antibody binding of this region across HAstV serotypes. While there is no overlap between the 3E8 and 2D9 epitopes, S564 (D566 in Spike 2) is found in both the 2D9 and PL-2 epitopes. Sequence conservation may indicate that 3E8, 2D9, and PL-2 obstruct a conserved functional site on the virus surface directly or by proximity. Demonstrated in the next example is that 3E8 and 2D9 neutralize HAstV8 by the same mechanism despite their fully separate epitopes.

### Example 5—Antibodies 3E8 and 2D9 Block Attachment of HAstV8 to Caco-2 Cells

First, it was confirmed that mAbs 3E8 and 2D9 reduce virus infectivity. Purified HAstV8 particles were pre-incubated with serial dilutions of the ascites fluids of either 3E8 or 2D9, added to Caco-2 cell monolayers, and washed. The next day, infected cells were detected by an immunoperoxidase focus-forming assay. Virus infectivity decreased with increasing amounts of antibody (FIG. 5A). To determine the neutralization mechanisms of 3E8 and 2D9, assessed was whether the antibodies could block attachment of the virus to the surface of Caco-2 cells. Purified HAstV8 particles were pre-incubated with serial dilutions of the ascites fluids of either 3E8 or 2D9, and the virus-antibody mix was added to Caco-2 cell monolayers on ice. At this temperature, the virus is able to attach to the cell surface but does not enter the cell. The unbound virus was removed by washing, total RNA was extracted, and the viral genome was quantified by RT-qPCR. As can be seen in FIG. 5B, both antibodies were able to efficiently impede HAstV8 attachment, reducing binding of the virus by more than 90% at a 1:2500 dilution of the ascites fluids, as compared to virus bound in the absence of antibody. As a negative control, mAb 3B4, specific for HAstV1 and previously shown not to neutralize HAstV8 infectivity, was added; no reduction of virus binding was observed with this antibody. This result shows a direct correlation between the inhibition of virus infectivity and of virus binding.

To confirm that mAbs 3E8 and 2D9 inhibit virus attachment directly and not through aggregation of virus particles, the ability of the antibodies to detach the virus once it has bound to the cell surface was evaluated. For this assay, purified HAstV8 was added to Caco-2 cells and incubated on ice. The unbound virus was removed by washing, and then serial 1:5 dilutions of the ascites fluids of 3E8, 2D9 and 3B4 were added to the cells and further incubated on ice. After this incubation, the cells were washed again, and the viral RNA was quantified as described above. As can be seen in FIG. 5C, both mAbs 2D9 and 3E8 (although 2D9 more efficiently) were able to detach the virus from the cell surface, presumably by competition of the antibody with the virus-receptor interaction. Negative control 3B4 was unable to detach the virus. Altogether, these results indicate that antibodies 3E8 and 2D9 neutralize virus infectivity by inhibiting the attachment of the virus to the cell surface.

### Example 6—Antibodies 3E8 and 2D9 Block Attachment of GFP-Spike 8 to Caco-2 Cells

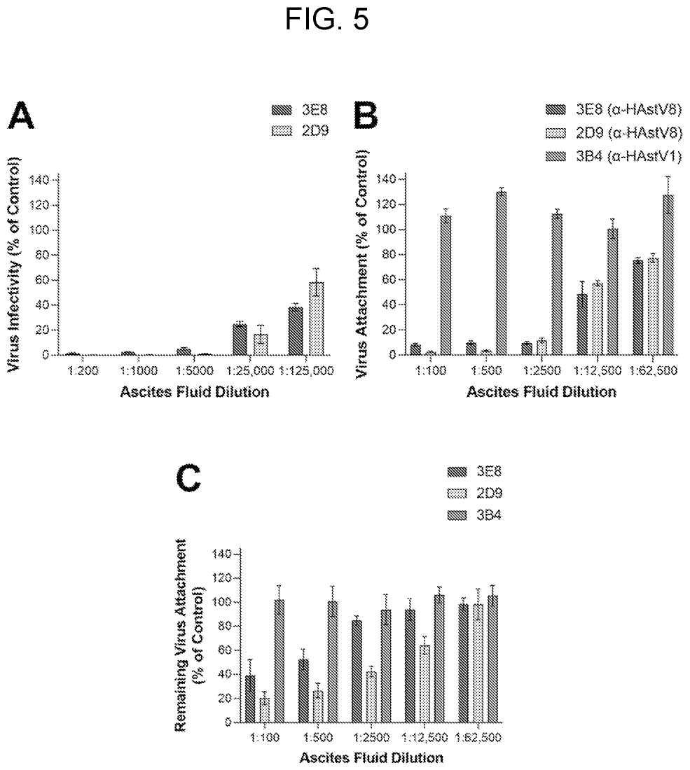

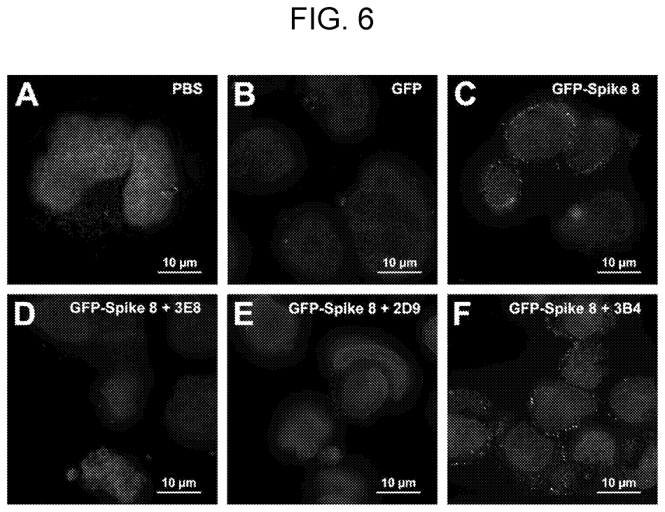

Finally, to test if the HAstV capsid spike alone is sufficient for cell attachment and to show that antibodies block this attachment, the capacity of a recombinantly expressed fluorescent GFP-Spike 8 fusion protein to attach to Caco-2 cells, and the ability of recombinant antigen binding fragments (Fabs) 3E8 and 2D9 to prevent this attachment, were examined. Fab constructs were chosen for this set of experiments to avoid the possible dimerization effect of mAbs and (sometimes) scFvs, which would potentially cause cross-linking issues with dimeric GFP-Spike 8. Caco-2 cells grown on glass coverslips were incubated with GFP alone, GFP-Spike 8 alone, or with GFP-Spike 8 and Fab 3E8, 2D9, or 3B4. Cells were washed, fixed, and imaged by fluorescence microscopy. As shown in FIG. 6, Caco-2 cells incubated with phosphate-buffered saline (PBS) alone or GFP alone had little to no green signal. In contrast, Caco-2 cells incubated with GFP-Spike 8 had bright green punctate patterns that appear at or near the cell membranes. Furthermore, cells incubated with GFP-Spike 8 and Fab 3E8 or Fab 2D9 had minimal puncta, suggesting specific blocking of Spike 8 attachment to cells by the Fabs. The negative control Fab 3B4 did not block attachment. These data reveal that the human astrovirus capsid spike alone is sufficient for cell attachment and that antibodies 3E8 and 2D9 are able to preclude virus attachment by specifically blocking the spike.

### Example 7—Generation and Sequencing of Human Astrovirus-Neutralizing Monoclonal Antibodies 2D9, 3B4, 3E8, 3H4, 4B6, 2A2, and 7C8

Human astrovirus (HAstV) is a leading cause of viral gastroenteritis in children, with ˜3.9 million cases of HAstV gastroenteritis per year in the United States alone. Worldwide, HAstV accounts for 7-23% of viral gastroenteritis infections in children. Astroviruses can also cause severe, chronic, and/or systemic infections in immune-compromised patients. No licensed vaccines or antiviral therapies exist for HAstV infection.

While HAstV is normally associated with acute gastroenteritis infection in children, many studies have identified HAstV cases causing severe, chronic, and/or systemic infections in immune-compromised patients. HAstV has often been associated with nosocomial infections in hospitals. In particular, chronic or severe HAstV infections have been observed in bone marrow recipients, AIDS patients, and other immune-compromised patients. In addition, several cases of neurovirulent human astrovirus infections have been reported, most of which were attributed to HAstV strains in the divergent HAstV-VA1 clade. These infections are associated with encephalitis or meningitis in immune-compromised patients.

Several lines of evidence suggest that antibodies developed by the adaptive immune response are important in protection against HAstV infection. First, the rarity of HAstV infection in adults suggests that they have developed a protective adaptive immune response during childhood. In fact, over 70% of healthy adults have anti-HAstV antibodies. Furthermore, two HAstV clinical studies with healthy volunteers found that those with more severe diarrheal disease had no detectable anti-HAstV antibodies. Finally, immunoglobulin therapy was associated with the recovery of an immune-compromised patient with severe and persistent HAstV infection. Together, these data suggest that a monoclonal antibody drug that neutralizes HAstV will provide an effective treatment for HAstV infection and may also be used to reduce the risk of HAstV infection in high-risk individuals.

Because standard methods using recombinant DNA technology would be used to produce a mouse-human chimeric monoclonal antibody that neutralizes HAstV, these antibody drugs are expected to be highly safe for immune-compromised individuals, and would have low side effects, low toxicity, and require infrequent dosing. The humanized monoclonal antibodies could be used in one of two ways: (1) as a preventative solution for high-risk individuals during a hospital HAstV outbreak or during Winter/Spring months when HAstV infection rates are highest, or (2) as a therapeutic solution for immune-compromised individuals with chronic and/or severe HAstV infections.

To generate monoclonal antibodies, mice were immunized with recombinant human astrovirus (HAstV)-1, -2, and -8 capsid spike proteins to make mAbs 2D9, 3B4, 3E8, 3H4, and 41B6 (doi.org/10.1128/jvi.01465-18). Mice were immunized with live HAstV-VA1 virus to make mAbs 2A2 and 708.

After immunization, mouse hybridoma cell lines were generated. ELISAs were used to identify antigen-specific mouse monoclonal antibodies, and HAstV-neutralization assays were used to identify those monoclonal antibodies with virus-neutralizing activity. Hybridoma cell mRNAs were extracted, cDNAs were generated, and antibody heavy and light chain amino acid sequences were determined (doi.org/1 0.1371/journal.pone.021 8717).

Monoclonal antibody (mAb) sequences (with signal sequences removed) are provided in Table 3 below. Variable regions are in bold and CDRs (according to Kabat) are underlined. For the single-chain fragment variable (scFv) sequences, the mAb heavy variable heavy region is fused to a flexible linker (indicated by italics) and then to the mAb light variable region. For the full-length mAb sequences, mouse variable region sequences are fused to the constant region sequences from human anti-HIV IgG1 antibody VRC01 (doi.org/1 0.11 26/science.1192819).

TABLE 3
Monoclonal Antibody Amino Acid Sequences

| | |
|---|---|
| Anti-HAstV-8 mAb 2D9 heavy | QVQLKQSGPGLVQPSQSLSITCTVSGFSLTSYGVHWVRQ |
| chain (mouse heavy chain | SPGKGLEWLGVIWSGGSTDYNAAFISRLSISKDNSKSQV |
| variable fused to human heavy | FFKMNSLQANDTAIYYCARNSLLDAMDYWGQGTSVTVS |
| chain constant) | SASTKGPSVFPLAPSSKSTSGGTAALGCLVKDYFPEPVTV |
| V_{H} (bold)-SEQ ID NO: 1 | SWNSGALTSGVHTFPAVLQSSGLYSLSSVVTVPSSSLGT |
| V_{H} CDR1-SEQ ID NO: 2 | QTYICNVNHKPSNTKVDKKVEPKSCDKTHTCPPCPAPELL |
| V_{H} CDR2-SEQ ID NO: 3 | GGPSVFLFPPKPKDTLMISRTPEVTCVVVDVSHEDPEVKF |
| V_{H} CDR3-SEQ ID NO: 4 | NWYVDGVEVHNAKTKPREEQYNSTYRVVSVLTVLHQDW |
| | LNGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 56) |
| Anti-HAstV-8 mAb 2D9 kappa | SIVMTQTPKFLLVSAGDRVTITCKASQSVSNAVAWYQQK |
| chain (mouse light chain | PGQSPKLLIYYASNRYTGVPDRFTGSGYGTDFTFTISTVQ |
| variable fused to human light | AEDLAVYFCQQDYSSPLTFGAGTKLELKRRTVAAPSVFIF |
| chain constant, CDRs) | PPSDEQLKSGTASVVCLLNNFYPREAKVQWKVDNALQSG |
| V_{L} (bold)-SEQ ID NO: 5 | NSQESVTEQDSKDSTYSLSSTLTLSKADYEKHKVYACEVT |
| V_{L} CDR1-SEQ ID NO: 6 | HQGLSSPVTKSFNRGEC (SEQ ID NO: 57) |
| V_{L} CDR2-SEQ ID NO: 7 | |
| V_{L} CDR3-SEQ ID NO: 8 | |
| Anti-HAstV-8 scFv 2D9 (mouse | QVQLKQSGPGLVQPSQSLSITCTVSGFSLTSYGVHWVRQ |
| heavy chain variable, linker, | SPGKGLEWLGVIWSGGSTDYNAAFISRLSISKDNSKSQVF |
| mouse light chain variable) | FKMNSLQANDTAIYYCARNSLLDAMDYWGQGTSVTVSSG |
| | GSGGGGSGGGGSGGGGSSIVMTQTPKFLLVSAGDRVTIT |
| | CKASQSVSNAVAWYQQKPGQSPKLLIYYASNRYTGVPDR |
| | FTGSGYGTDFTFTISTVQAEDLAVYFCQQDYSSPLTFGAG |
| | TKLELKR (SEQ ID NO: 58) |
| Anti-HAstV-1 3B4 heavy chain | QVQLHQPGAELVKPGASVNLSCKASGYTFTSYWMHWV |
| (mouse heavy chain variable | KQRPGQGLEWIGEINPSSGRANYNEKFKNKATLTVDKSS |
| fused to human heavy chain | ITAYMHLSSLTSEDSAVYYCHWDYYAMDYWGQGTSVTV |
| constant) | SSASTKGPSVFPLAPSSKSTSGGTAALGCLVKDYFPEPVT |
| V_{H} (bold)-SEQ ID NO: 9 | VSWNSGALTSGVHTFPAVLQSSGLYSLSSVVTVPSSSLGT |
| V_{H} CDR1-SEQ ID NO: 10 | QTYICNVNHKPSNTKVDKKVEPKSCDKTHTCPPCPAPELL |
| V_{H} CDR2-SEQ ID NO: 11 | GGPSVFLFPPKPKDTLMISRTPEVTCVVVDVSHEDPEVKF |
| V_{H} CDR3-SEQ ID NO: 12 | NWYVDGVEVHNAKTKPREEQYNSTYRVVSVLTVLHQDW |
| | LNGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 59) |
| Anti-HAstV-1 3B4 kappa chain | DIVLTQSPATLSVTPGDSVSLSCRASQSISNNLHWYQQK |
| (mouse light chain variable | SHESPRLLFKSASQSISGIPSRFSGSGSGTDFTLSINSVET |
| fused to human light chain | EDFGMYFCQQTNSWPLTFGTGTKLDLKRRTVAAPSVFIF |
| constant) | PPSDEQLKSGTASVVCLLNNFYPREAKVQWKVDNALQSG |
| V_{L} (bold)-SEQ ID NO: 13 | NSQESVTEQDSKDSTYSLSSTLTLSKADYEKHKVYACEVT |
| V_{L} CDR1-SEQ ID NO: 14 | HQGLSSPVTKSFNRGEC (SEQ ID NO: 60) |
| V_{L} CDR2-SEQ ID NO: 15 | |
| V_{L} CDR3-SEQ ID NO: 16 | |
| Anti-HAstV-1 scFv 3B4 (mouse | QVQLHQPGAELVKPGASVNLSCKASGYTFTSYWMHWVK |
| heavy chain variable, linker, | QRPGQGLEWIGEINPSSGRANYNEKFKNKATLTVDKSSIT |
| mouse light chain variable) | AYMHLSSLTSEDSAVYYCHWDYYAMDYWGQGTSVTVSS |
| | GGSGGGGSGGGGSGGGGSDIVLTQSPATLSVTPGDSVS |
| | LSCRASQSISNNLHWYQQKSHESPRLLFKSASQSISGIPS |
| | RFSGSGSGTDFTLSINSVETEDFGMYFCQQTNSWPLTFG |
| | TGTKLDLKR (SEQ ID NO: 61) |
| Anti-HAstV-8 3E8 heavy chain | DVQLQESGPGLVKPSQSLSLTCSVTGYSITSGYYWNWIR |
| (mouse heavy chain variable | QFPGNKLEWMGYISYDGSNNYNPSLKNRISITRDTSKNQ |
| fused to human heavy chain | FFLKLNSVTTEDTATYYCATFYDGYDYWGQGTTLTVSSA |
| constant, CDRs) | STKGPSVFPLAPSSKSTSGGTAALGCLVKDYFPEPVTVS |
| V_{H} (bold)-SEQ ID NO: 17 | WNSGALTSGVHTFPAVLQSSGLYSLSSVVTVPSSSLGTQ |
| V_{H} CDR1-SEQ ID NO: 18 | TYICNVNHKPSNTKVDKKVEPKSCDKTHTCPPCPAPELLG |
| V_{H} CDR2-SEQ ID NO: 19 | GPSVFLFPPKPKDTLMISRTPEVTCVVVDVSHEDPEVKFN |
| V_{H} CDR3-SEQ ID NO: 20 | WYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 62) |
| Anti-HAstV-8 3E8 kappa chain | DIVMTQSHKFMSTSVGDRVSITCKASQDVSTAVAWYQQK |
| (mouse light chain variable | PGQSPKLLIYWASTRHTGVPDRFTGSGSGTDYTLTISSVQ |
| fused to human light chain | AEDLALYYCQQHYSTPFTFGSGTKLEIKRRTVAAPSVFIF |
| constant, CDRs) | PPSDEQLKSGTASVVCLLNNFYPREAKVQWKVDNALQSG |
| V_{L} (bold)-SEQ ID NO: 21 | NSQESVTEQDSKDSTYSLSSTLTLSKADYEKHKVYACEVT |
| V_{L} CDR1-SEQ ID NO: 22 | HQGLSSPVTKSFNRGEC (SEQ ID NO: 63) |
| V_{L} CDR2-SEQ ID NO: 23 | |
| V_{L} CDR3-SEQ ID NO: 24 | |
| Anti-HAstV-8 scFv 3E8 (mouse | DVQLQESGPGLVKPSQSLSLTCSVTGYSITSGYYWNWIR |
| heavy chain variable, linker, | QFPGNKLEWMGYISYDGSNNYNPSLKNRISITRDTSKNQF |
| mouse light chain variable, | FLKLNSVTTEDTATYYCATFYDGYDYWGQGTTLTVSSGG |
| CDRs) | SGGGGSGGGGSGGGGSDIVMTQSHKFMSTSVGDRVSIT |
| | CKASQDVSTAVAWYQQKPGQSPKLLIYWASTRHTGVPDR |
| | FTGSGSGTDYTLTISSVQAEDLALYYCQQHYSTPFTFGSG |
| | TKLEIKR (SEQ ID NO: 64) |
| Anti-HAstV-1 3H4 heavy chain | QVQLKESGPGLVAPSQSLSISCTVSGFSLTTFGIHWIRQP |
| (mouse heavy chain variable | PGKGLEWLGVIWAAGTTNYNSTLKSRLTITKDNSRSQVF |
| fused to human heavy chain | LKMNSLQTYDTAIYYCVREDYDYFFGLDYWGQGTSVTVS |
| constant) | SASTKGPSVFPLAPSSKSTSGGTAALGCLVKDYFPEPVTV |
| V_{H} (bold)-SEQ ID NO: 25 | SWNSGALTSGVHTFPAVLQSSGLYSLSSVVTVPSSSLGT |
| V_{H} CDR1-SEQ ID NO: 26 | QTYICNVNHKPSNTKVDKKVEPKSCDKTHTCPPCPAPELL |
| V_{H} CDR2-SEQ ID NO: 27 | GGPSVFLFPPKPKDTLMISRTPEVTCVVVDVSHEDPEVKF |
| V_{H} CDR3-SEQ ID NO: 28 | NWYVDGVEVHNAKTKPREEQYNSTYRVVSVLTVLHQDW |
| | LNGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 65) |
| Anti-HAstV-1 3H4 lambda | QAVVTQESALTTSPGETVTLTCRSSTGAVTTSNYASWVQ |
| chain (mouse light chain | EKPDHLFIGLIGGTNNRAPGVPARFSGSLIGDKAALTITG |
| variable fused to human light | AQTDDEAIYFCALWFSNHWVFGGGTKLTVLGRTVAAPSV |
| chain constant) | FIFPPSDEQLKSGTASVVCLLNNFYPREAKVQWKVDNALQ |
| V_{L} (bold)-SEQ ID NO: 29 | SGNSQESVTEQDSKDSTYSLSSTLTLSKADYEKHKVYACE |
| V_{L} CDR1-SEQ ID NO: 30 | VTHQGLSSPVTKSFNRGEC (SEQ ID NO: 66) |
| V_{L} CDR2-SEQ ID NO: 31 | |
| V_{L} CDR3-SEQ ID NO: 32 | |
| Anti-HAstV-1 scFv 3H4 (heavy | QVQLKESGPGLVAPSQSLSISCTVSGFSLTTFGIHWIRQPP |
| chain variable, linker, light | GKGLEWLGVIWAAGTTNYNSTLKSRLTITKDNSRSQVFLK |
| chain variable) | MNSLQTYDTAIYYCVREDYDYFFGLDYWGQGTSVTVSSG |
| | GSGGGGSGGGGSGGGGSQAVVTQESALTTSPGETVTLT |
| | CRSSTGAVTTSNYASWVQEKPDHLFIGLIGGTNNRAPGVP |
| | ARFSGSLIGDKAALTITGAQTDDEAIYFCALWFSNHWVFG |
| | GGTKLTVLG (SEQ ID NO: 67) |
| Anti-HAstV-4 4B6 heavy chain | QVQLKESGPGLVAPSQSLSITCTVSGFSLTSYGVHWVRQ |
| (mouse heavy chain variable | PPGKGLEWLGVIWADGSTNYNSALMSRLSISKDNSKSQ |
| fused to human heavy chain | VFLKMNSLQTDDTAMYYCARWTYGDYFDYWGQGTTLTV |
| constant) | SSASTKGPSVFPLAPSSKSTSGGTAALGCLVKDYFPEPVT |
| V_{H} (bold)-SEQ ID NO: 33 | VSWNSGALTSGVHTFPAVLQSSGLYSLSSVVTVPSSSLGT |
| V_{H} CDR1-SEQ ID NO: 1 | QTYICNVNHKPSNTKVDKKVEPKSCDKTHTCPPCPAPELL |
| V_{H} CDR2-SEQ ID NO: 34 | GGPSVFLFPPKPKDTLMISRTPEVTCVVVDVSHEDPEVKF |
| V_{H} CDR3-SEQ ID NO: 35 | NWYVDGVEVHNAKTKPREEQYNSTYRVVSVLTVLHQDW |
| | LNGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 68) |
| Anti-HAstV-2 4B6 kappa chain | DIQMTQTTSSLSASLGDRVTISCSASQGISNYLNWYQQK |
| (mouse light chain variable | PDGTVKLLIYYTSSLHSGVPSRFSGSGSGTDYSLTISNLE |
| fused to human light chain | PEDIATYYCQQYSKLPYTFGGGTKLEIKRRTVAAPSVFIFP |
| constant) | PSDEQLKSGTASVVCLLNNFYPREAKVQWKVDNALQSGN |
| V_{L} (bold)-SEQ ID NO: 36 | SQESVTEQDSKDSTYSLSSTLTLSKADYEKHKVYACEVTH |
| V_{L} CDR1-SEQ ID NO: 37 | QGLSSPVTKSFNRGEC (SEQ ID NO: 69) |
| V_{L} CDR2-SEQ ID NO: 38 | |
| V_{L} CDR3-SEQ ID NO: 39 | |
| Anti-HAstV-2 scFv 4B6 (heavy | QVQLKESGPGLVAPSQSLSITCTVSGFSLTSYGVHWVRQ |
| chain variable, linker, light | PPGKGLEWLGVIWADGSTNYNSALMSRLSISKDNSKSQV |
| chain variable) | FLKMNSLQTDDTAMYYCARWTYGDYFDYWGQGTTLTVS |
| | SGGSGGGGSGGGGSGGGGSDIQMTQTTSSLSASLGDRV |
| | TISCSASQGISNYLNWYQQKPDGTVKLLIYYTSSLHSGVPS |
| | RFSGSGSGTDYSLTISNLEPEDIATYYCQQYSKLPYTFGG |
| | GTKLEIKR (SEQ ID NO: 70) |
| Anti-HAstV-VA1 2A2 heavy | DVQLVESGGGLVQPGGSRKLSCAASGFTFSYFGMHWV |
| chain (mouse heavy chain | RQAPEKGLEWVAYISSGSNTIYYADTVKGRFTISRDNPKN |
| variable fused to human heavy | TLFLQMTSLRSEDTAMYYCARAYYGNHYYAMDFWGPGT |
| chain constant) | SVTVSSASTKGPSVFPLAPSSKSTSGGTAALGCLVKDYFP |
| V_{H} (bold)-SEQ ID NO: 40 | EPVTVSWNSGALTSGVHTFPAVLQSSGLYSLSSVVTVPSS |
| V_{H} CDR1-SEQ ID NO: 41 | SLGTQTYICNVNHKPSNTKVDKKVEPKSCDKTHTCPPCPA |
| V_{H} CDR2-SEQ ID NO: 42 | PELLGGPSVFLFPPKPKDTLMISRTPEVTCVVVDVSHEDP |
| V_{H} CDR3-SEQ ID NO: 43 | EVKFNWYVDGVEVHNAKTKPREEQYNSTYRVVSVLTVLH |
| | QDWLNGKEYKCKVSNKALPAPIEKTISKAKGQPREPQVYT |
| | LPPSRDELTKNQVSLTCLVKGFYPSDIAVEWESNGQPEN |
| | NYKTTPPVLDSDGSFFLYSKLTVDKSRWQQGNVFSCSVM |
| | HEALHNHYTQKSLSLSPGK (SEQ ID NO: 71) |
| Anti-HAstV-VA1 2A2 kappa | DIQMTQSPASLSVSVGETVTITCRASENIYSNLAWYQQKR |
| chain (mouse light chain | GKSPQLLVHAARDLATGVPSRFSGSGSGTQYSLKINSLQ |
| variable fused to human light | SEDFGTYYCQHFWETPWTFGGGTKLEIKRRTVAAPSVFIF |
| chain constant) | PPSDEQLKSGTASVVCLLNNFYPREAKVQWKVDNALQSG |
| V_{L} (bold)-SEQ ID NO: 44 | NSQESVTEQDSKDSTYSLSSTLTLSKADYEKHKVYACEVT |
| V_{L} CDR1-SEQ ID NO: 45 | HQGLSSPVTKSFNRGEC (SEQ ID NO: 72) |
| V_{L} CDR2-SEQ ID NO: 46 | |
| V_{L} CDR3-SEQ ID NO: 47 | |
| Anti-HAstV-VA1 scFv 2A2 | DVQLVESGGGLVQPGGSRKLSCAASGFTFSYFGMHWVR |
| (heavy chain variable, linker, | QAPEKGLEWVAYISSGSNTIYYADTVKGRFTISRDNPKNTL |
| light chain variable) | FLQMTSLRSEDTAMYYCARAYYGNHYYAMDFWGPGTSV |
| | TVSSGGSGGGGSGGGGSGGGGSDIQMTQSPASLSVSV |
| | GETVTITCRASENIYSNLAWYQQKRGKSPQLLVHAARDLA |
| | TGVPSRFSGSGSGTQYSLKINSLQSEDFGTYYCQHFWET |
| | PWTFGGGTKLEIKR (SEQ ID NO: 73) |
| Anti-HAstV-VA1 7C8 heavy | EVQLVESGGGLVKPGGSLKLSCAASGFTFSDYYMYWIR |
| chain (mouse heavy chain | QTPEKRLEWVATISDGGFSTYYPDSVKGRFTISRDNAKN |
| variable fused to human heavy | NLFLQMSSLKSEDTAIYYCARFGAYSTFWGQGTLVTVSS |
| chain constant) | ASTKGPSVFPLAPSSKSTSGGTAALGCLVKDYFPEPVTVS |
| V_{H} (bold)-SEQ ID NO: 48 | WNSGALTSGVHTFPAVLQSSGLYSLSSVVTVPSSSLGTQ |
| V_{H} CDR1-SEQ ID NO: 59 | TYICNVNHKPSNTKVDKKVEPKSCDKTHTCPPCPAPELLG |
| V_{H} CDR2-SEQ ID NO: 50 | GPSVFLFPPKPKDTLMISRTPEVTCVVVDVSHEDPEVKFN |
| V_{H} CDR3-SEQ ID NO: 51 | WYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKT |
| | TPPVLDSDGSFFLYSKLTVDKSRWQQGNVFSCSVMHEAL |
| | HNHYTQKSLSLSPGK (SEQ ID NO: 74) |
| Anti-HAstV-VA1 kappa | QIVLSQSPAILSASPGEKVTMTCRASSSVSYMYWYQQKP |
| chain (mouse light chain | GSSPKPWIYATSNLASGVPARFSGSGSGTSYSLTISRVE |
| variable fused to human light | AEDAATYYCQQWSSDPPMTFGGGTKLEIKRRTVAAPSVF |
| chain constant) | IFPPSDEQLKSGTASVVCLLNNFYPREAKVQWKVDNALQS |
| V_{L} (bold)-SEQ ID NO: 52 | GNSQESVTEQDSKDSTYSLSSTLTLSKADYEKHKVYACEV |
| V_{L} CDR1-SEQ ID NO: 53 | THQGLSSPVTKSFNRGEC (SEQ ID NO: 75) |
| V_{L} CDR2-SEQ ID NO: 54 | |
| V_{L} CDR3-SEQ ID NO: 55 | |
| Anti-HAstV-VA1 scFv 7C8 | EVQLVESGGGLVKPGGSLKLSCAASGFTFSDYYMYWIRQ |
| (heavy chain variable, linker, | TPEKRLEWVATISDGGFSTYYPDSVKGRFTISRDNAKNNL |
| light chain variable) | FLQMSSLKSEDTAIYYCARFGAYSTFWGQGTLVTVSSGG |
| | SGGGGSGGGGSGGGGSQIVLSQSPAILSASPGEKVTMTC |
| | RASSSVSYMYWYQQKPGSSPKPWIYATSNLASGVPARFS |
| | GSGSGTSYSLTISRVEAEDAATYYCQQWSSDPPMTFGGG |
| | TKLEIKR (SEQ ID NO: 76) |

### Discussion

Demonstrated herein is that immunization with recombinant capsid spike elicits high-affinity antibodies that neutralize the virus, making the spike a candidate antigen for a subunit vaccine. Also demonstrated herein is that there are at least two distinct epitopes on the HAstV capsid spike surface. Antibodies targeting either one of these epitopes neutralize the virus by inhibiting virus attachment to cells. The significant overlap between the epitopes targeted by PL-2 on Spike 2 and 3E8 on Spike 8 shows that antibodies can target the same epitope on spikes from two HAstV serotypes and neutralize the virus by the same mechanism, blocking attachment to cells. This epitope intersection indicates that functional regions on the spike surface are likely conserved across HAstV serotypes, despite a high degree of sequence variability in the spike, which has 41-76% sequence identity between all 8 classical serotypes.⁷

The structures of scFv 3E8/Spike 8 and scFv 2D9/Spike 8 illustrate that the immune response can target the spike of a single serotype at different epitopes. This targeting mechanism reveals that spikes used as immunogens elicit a neutralizing polyclonal response to human astrovirus. A polyclonal response is beneficial in generating long-term protection against infection because it minimizes virus neutralization escape by mutations. Indeed, the HAstV8 escape mutant to 3E8 is still susceptible to neutralization by 2D9, and vice versa.⁴¹ Therefore, immunization with spikes presents a robust strategy for HAstV subunit vaccine immunogen design. Importantly, these results also indicate that obstructing any one of multiple sites on the spike surface can prevent virus infectivity. Consequently, HAstV has several weak points that may be exploited individually or in combination in an antiviral therapy.

### Materials and Methods

Expression and Purification of scFv 3E8

Mouse hybridoma cells producing mAb 3E8 were generated as reported in⁴¹. The amino acid sequences of the mAb 3E8 variable regions were identified as described in⁴⁶, allowing for recombinant antibody expression. A synthetic gene codon-optimized for Drosophila melanogaster containing the 3E8 kappa chain variable region connected to the 3E8 heavy chain variable region by a GGS(GGGGS)₃ (SEQ ID NO:77) linker was purchased from Integrated DNA Technologies. This gene was cloned into the pCMV-VRC01 vector by Gibson assembly in frame with an N-terminal secretion signal sequence and a C-terminal thrombin cleavage site followed by a Twin-Strep-tag. The resulting pCMV-VRC01_scFv_3E8 expression plasmid was used to electroporate Chinese Hamster Ovary suspension (CHO-S) cells using the MaxCyte system. The scFv 3E8 was expressed for 8 days by CHO-S cells growing in CD OptiCHO expression medium (Gibco) supplemented with 1 mM sodium butyrate (Sigma-Aldrich), 8 mM L-glutamine (Gibco), 1×HT supplement (Gibco), and 0.1% Pluronic F68 (Gibco) at 32° C. with 125 rpm shaking. Every 24 hours, cells were fed with CHO CD EfficientFeed A (Gibco) supplemented with 7 mM L-glutamine (Gibco), 5.5% glucose (Sigma Aldrich), and 23.4 g/L yeastolate (BD). After 8 days, cells were pelleted and medium containing secreted scFv 3E8 was 0.22-μM filtered, buffered to Strep wash buffer (50 mM Tris-HCl pH 8.0, 150 mM NaCl, 1 mM EDTA), and supplemented with BioLock (IBA Lifesciences) to mask free biotin in the medium. The sample was 0.22-μM filtered again, affinity purified on two tandem 5 mL StrepTrap HP columns (GE), and eluted in Strep elution buffer (Strep wash buffer with 2.5 mM desthiobiotin). The scFv 3E8 was dialyzed overnight into 10 mM Tris-HCl pH 7.5, 150 mM NaCl (TBS pH 7.5).

Expression and Purification of scFv 3E8

Mouse hybridoma cells producing mAb 3E8 were generated as reported in⁴¹. The amino acid sequences of the mAb 3E8 variable regions were identified as described in⁴⁶, allowing for recombinant antibody expression. A synthetic gene codon-optimized for Drosophila melanogaster containing the 3E8 kappa chain variable region connected to the 3E8 heavy chain variable region by a GGS(GGGGS)₃ (SEQ ID NO:77) linker was purchased from Integrated DNA Technologies. This gene was cloned into the pCMV-VRC01 vector by Gibson assembly in frame with an N-terminal secretion signal sequence and a C-terminal thrombin cleavage site followed by a Twin-Strep-tag. The resulting pCMV-VRC01_scFv_3E8 expression plasmid was used to electroporate Chinese Hamster Ovary suspension (CHO-S) cells using the MaxCyte system. The scFv 3E8 was expressed for 8 days by CHO-S cells growing in CD OptiCHO expression medium (Gibco) supplemented with 1 mM sodium butyrate (Sigma-Aldrich), 8 mM L-glutamine (Gibco), 1×HT supplement (Gibco), and 0.1% Pluronic F68 (Gibco) at 32° C. with 125 rpm shaking. Every 24 hours, cells were fed with CHO CD EfficientFeed A (Gibco) supplemented with 7 mM L-glutamine (Gibco), 5.5% glucose (Sigma Aldrich), and 23.4 g/L yeastolate (BD). After 8 days, cells were pelleted and medium containing secreted scFv 3E8 was 0.22-μM filtered, buffered to Strep wash buffer (50 mM Tris-HCl pH 8.0, 150 mM NaCl, 1 mM EDTA), and supplemented with BioLock (IBA Lifesciences) to mask free biotin in the medium. The sample was 0.22-μM filtered again, affinity purified on two tandem 5 mL StrepTrap HP columns (GE), and eluted in Strep elution buffer (Strep wash buffer with 2.5 mM desthiobiotin). The scFv 3E8 was dialyzed overnight into 10 mM Tris-HCl pH 7.5, 150 mM NaCl (TBS pH 7.5).

Expression and Purification of scFv 2D9

Mouse hybridoma cells producing mAb 2D9 were generated as reported in⁴¹. The amino acid sequences of the mAb 2D9 variable regions were identified as described in⁴⁶, allowing for recombinant antibody expression. A synthetic gene codon-optimized for Drosophila melanogaster containing the 2D9 kappa chain variable region connected to the 2D9 heavy chain variable region by a GGS(GGGGS)₃ (SEQ ID NO:77) linker and flanked by BgIII and NheI restriction sites was purchased from Integrated DNA Technologies. This gene was cloned into the pMT_puro_BiP vector by restriction digest in frame with an N-terminal BiP secretion signal sequence and a C-terminal thrombin cleavage site followed by a Twin-Strep-tag. The plasmid contains a metallothionein promoter for induction of gene expression as well as a puromycin-resistance gene. The resulting pMT_puro_BiP_scFv_2D9 expression plasmid was used to obtain stably transfected D. melanogaster Schneider 2 (S2) cells by transfection with FuGENE HD (Promega) followed by selection with 5 μg/mL puromycin. The S2 cells were grown in Schneider's S2 medium (Gibco) with 10% heat-inactivated FBS and 1× pen/strep (Gibco) at the selection stage. Cells were then adapted to serum-free, antibiotic-free ESF 921 medium (Expression Systems) for expression. The stable S2 cells were grown in shaker flasks to 3.0×10⁶ cells/mL. Expression of scFv 2D9 was induced with 500 μM CuCl₂ and cells were incubated at 27° C. with 125 rpm shaking. After 5 days, cells were pelleted and medium containing secreted scFv 2D9 was 0.22-μM filtered, buffered to Strep wash buffer (50 mM Tris pH 8.0, 150 mM NaCl, 1 mM EDTA) and concentrated 200-fold by tangential flow filtration. After supplementation with BioLock (IBA Lifesciences) to mask free biotin in the medium, the sample was 0.22-μM filtered again, affinity purified on two tandem 5 mL StrepTrap HP columns (GE), and eluted in Strep elution buffer (Strep wash buffer with 2.5 mM desthiobiotin). The scFv 2D9 was dialyzed overnight into 10 mM Tris-HCl pH 8.0, 150 mM NaCl (TBS pH 8.0).

### Expression and Purification of the Spike 8 Antigen

A synthetic gene codon-optimized for E. coli expression encoding the HAstV serotype 8 capsid spike protein amino acids 429 to 647 (Spike 8, UniProtKB entry Q91FX1) was purchased from Integrated DNA Technologies. To make the Spike 8 expression plasmid, the gene was cloned into pET52b (Addgene) under control of the T7 promoter in-frame with a C-terminal thrombin cleavage site and a 10-histidine purification tag. The plasmid was verified by DNA sequencing. Next, the plasmid was transformed into E. coli strain BL21(DE3). Cultures were inoculated and grown in LB/ampicillin medium. At an optical density of 0.6, protein production was induced with 1 mM isopropyl-D-thiogalactopyranoside (IPTG) at 18° C. for 18 hours. E. coli cells were lysed by ultrasonication in 20 mM Tris-HCl pH 8.0, 500 mM NaCl, and 20 mM imidazole (Buffer A) containing 2 mM MgCl₂, 0.0125 U/μL benzonase (Merck Millipore), and 1× protease inhibitor cocktail set V EDTA-free (Merck Millipore). The protein was batch purified from soluble lysates with TALON metal affinity resin (GE Healthcare) and eluted with Buffer A containing 500 mM imidazole. The protein was dialyzed overnight into 10 mM Tris-HCl pH 8.0, 150 mM NaCl (TBS pH 8.0) and further purified by size exclusion chromatography on a Superdex 75 column in TBS.

Biolayer Interferometry Affinity Determination of scFv 2D9 and scFv 3E8 for Spike 8

His-tagged Spike 8 was diluted to 0.5 μg/mL in BLI blocking buffer (PBS pH 7.4, 2% bovine serum albumin (BSA), 0.09% Tween-20). The scFv 3E8 was diluted to 80 nM (2.15 μg/mL) in BLI blocking buffer and five serial 1:2 dilutions were prepared. The scFv 2D9 was diluted to 20 nM (0.592 μg/mL) in BLI blocking buffer and five serial 1:2 dilutions were prepared. Using the 8-channel setting on an Octet® RED384 instrument (Fort6Bio), pre-equilibrated Anti-Penta-His (HIS1 K) sensor tips were dipped into the following solutions at 22° C. with shaking at 1000 rpm: First Baseline: BLI blocking buffer for 60 seconds, Loading: 0.5 μg/mL Spike 8 for 180 seconds, Second Baseline: BLI blocking buffer for 60 seconds, Association: 6 different concentrations of the scFv analyte in a serial 1:2 dilution for 60 seconds (scFv 3E8) or 120 seconds (scFv 2D9), and Dissociation: BLI blocking buffer for 120 seconds (scFv 3E8) or 180 seconds (scFv 2D9). A reference sample control was included in which 0 nM scFv was tested. A reference sensor control was also included in which 80 nM scFv 3E8 or 20 nM scFv 2D9 was associated to sensor tips that had not been pre-loaded with Spike 8 to test for non-specific scFv binding to the sensors. The sensors were dipped into BLI blocking buffer in these controls.

Each affinity determination experiment was performed in triplicate as independent assays. Data were processed separately and fit using the Octet® Data Analysis software v.7 (Fort6Bio). Before fitting, all datasets were reference-subtracted, aligned to the baseline, and aligned for inter-step correction through their respective dissociation steps as per the manufacturer's instructions. For each experiment, six different scFv analyte concentrations were used to fit association and dissociation globally using a 1:1 binding model. Ultimately, the goodness of fit was determined using R² and χ² values according to the manufacturer's guidelines. The reported K_{D}, R², and χ² values were averaged manually from the triplicate assays, generating the reported K_{D} standard deviation.

For the BLI competition assay, His-tagged Spike 8 was diluted to 0.5 μg/mL in BLI blocking buffer. The scFv 2D9 was diluted to 25 nM (0.74 μg/mL) in BLI blocking buffer. The scFv 3E8 was diluted to 400 nM (12 μg/mL) in BLI blocking buffer and used to prepare two serial 1:2 dilutions followed by one serial 1:4 dilution to obtain concentrations of 400, 200, 100, and 25 nM scFv 3E8. 0 nM scFv 3E8 was also tested. Pre-equilibrated Anti-Penta-His (HIS1 K) sensor tips were dipped into the following solutions at 22° C. with shaking at 1000 rpm: First Baseline: BLI blocking buffer for 60 seconds, Loading: 0.5 μg/mL Spike 8 for 180 seconds, Second Baseline: BLI blocking buffer for 60 seconds, scFv 2D9 Association: 25 nM scFv 2D9 for 120 seconds, scFv 3E8 Association: 5 different concentrations of scFv 3E8 for 120 seconds, and Dissociation: BLI blocking buffer for 180 seconds. A reference sensor control was included in which 25 nM scFv 2D9 followed by 400 nM scFv 3E8 were associated to sensor tips that had not been loaded with Spike 8 to test for non-specific scFv binding to the sensors. The sensors were dipped into BLI blocking buffer in this control. The dataset was reference-subtracted and aligned to the scFv 3E8 association step to generate FIG. 1C.

Formation and Structure Determination of the scFv 3E8/Spike 8 Complex

Complex formation was performed by incubation of 3 molar excess scFv 3E8 with Spike 8 overnight at 4° C. in TBS pH 7.2. Simultaneously, the purification tags from both scFv 3E8 and Spike 8 were removed by digestion of the complex with thrombin protease. The scFv 3E8/Spike 8 complex was purified by size exclusion chromatography on a Superdex 200 column in TBS pH 7.2. The complex coeluted at an apparent molecular mass of ˜105 kD compared to gel filtration standards, consistent with a 2:2 (scFv 3E8:Spike 8) complex in solution (data not shown). The purified complex was concentrated to 6.33 mg/mL. Hanging drops (2 μL) were formed by a 1:1 addition of concentrated protein complex and a well solution of 0.2 M lithium citrate tribasic and 18% PEG 3350. Crystals were grown by hanging drop vapor diffusion at 22° C. Crystals were transferred into a cryoprotectant solution of 0.2 M lithium citrate tribasic, 18.9% PEG 3350, and 25% glycerol and flash frozen in liquid nitrogen. Diffraction data from a single crystal were collected at cryogenic temperature at the Advanced Photon Source on beamline 23-ID-D using a wavelength of 1.033184 Å. The data were processed with Mosflm (ccp4i) and scaled with Aimless (ccp4i). The structure was solved by molecular replacement using Phenix.

Formation and Structure Determination of the scFv 2D9/Spike 8 Complex

Complex formation was performed by incubation of 3.5 molar excess scFv 2D9 with Spike 8 overnight at 4° C. in TBS pH 8.0. Simultaneously, the purification tags from both scFv 2D9 and Spike 8 were removed by digestion of the complex with thrombin protease. The scFv 2D9/Spike 8 complex was purified by size exclusion chromatography on a Superdex 200 column in TBS pH 8.0. The complex coeluted at an apparent molecular mass of ˜105 kD compared to gel filtration standards, consistent with a 2:2 (scFv 2D9:Spike 8) complex in solution (data not shown). The purified complex was concentrated to 3 mg/mL. Hanging drops (2 μL) were formed by a 1:1 addition of concentrated protein complex and a well solution of 0.1 M ammonium acetate, 0.1 M Bis-Tris pH 5.5, and 16% PEG 10,000. Crystals were grown by hanging drop vapor diffusion at 22° C. Crystals were transferred into a cryoprotectant solution of 0.1 M ammonium acetate, 0.1 M Bis-Tris pH 5.5, 16.8% PEG 10,000, and 25% ethylene glycol and flash frozen in liquid nitrogen. Six diffraction datasets from a single crystal were collected at cryogenic temperature at the Advanced Light Source on Beamline 8.3.1 using a wavelength of 1.115830 Å. The six datasets were processed separately with XDS and scaled together with Aimless (ccp4i). The structure was solved by molecular replacement using Phenix.

### HAstV8 Infectivity Neutralization Assay

Mouse mAbs 3E8 and 2D9 were raised in mice, hybridomas were generated, and ascites fluids were collected as described in⁴¹ Serial 1:5 dilutions of the ascites fluids for 3E8 or 2D9 were pre-incubated with HAstV8 particles (MOI=30) purified as reported previously⁴⁷ for 1 h at room temperature. To determine the titer of the non-neutralized infectious virus, confluent Caco-2 cell monolayers grown in 96-well plates were incubated with the virus-mAb complex for 1 h at 37° C. After this time, the cells were washed twice with PBS and were further incubated for 16 h at 37° C. The infected cells were detected by an immunoperoxidase focus-forming assay as described previously.⁴⁸ The remaining percent infectivity was determined by comparing the titer of infectious virus in the virus-mAb mix as compared to that obtained in the absence of antibodies, which was considered as 100%. Each point was performed in duplicate.

### HAstV8 Attachment Inhibition Assay

Serial 1:5 dilutions of the ascites fluids for 3E8 or 2D9 were pre-incubated with infectious HAstV8 particles (MOI=30) for 1 h at room temperature. Caco-2 cell monolayers grown in 48-well plates were washed once with PBS, and blocking solution (1% BSA in PBS) was added for 45 min at room temperature followed by a 15 min incubation on ice. The cells were then washed once with ice cold PBS and incubated with the virus-antibody complex for 1 h on ice. As a negative control, mAb 3B4 against HAstV1 was used. The unbound virus was washed three times with cold PBS, and the total RNA was extracted with TRIzol reagent (Invitrogen) according to the manufacturer's instructions. Viral RNA or cellular 18S RNA was reverse transcribed using MMLV reverse transcriptase (Invitrogen). RT-qPCR was performed with the pre-mixed reagent Real Q Plus Master Mix Green (Ampliqon), and the PCR reaction was carried out in an ABI Prism 7500 detection system (Applied Biosystems). The primers used to detect HAstV8 RNA were forward primer 5′ atgaattattttgatactgaggaagattacttggaa 3′ (SEQ ID NO:78) and reverse primer 5′ ctttcttgagaaatagataccaaagtacttcag 3′ (SEQ ID NO:79) (ORF 1b). For normalization, 18S ribosomal cellular RNA was amplified and quantified using forward primer 5′ cgaaagcatttgccaagaat 3′ (SEQ ID NO:80) and reverse primer 5′ gcatcgtttatggtcggaac 3′ (SEQ ID NO:81). The arithmetic means and standard deviation error bars from four independent experiments, performed in duplicate (3E8 and 2D9) or as single replicas (3B4) are shown. A Mann-Whitney U test was used to compare samples with mAbs 3E8 or 2D9 to samples with control mAb 3B4.

### HAstV8 Detachment Assay

Confluent Caco-2 cell monolayers in 48-well plates were blocked with 1% BSA in PBS for 45 min at room temperature followed by a 15 min incubation on ice. Purified HAstV8 viral particles were added at an MOI of 30 and incubated for 1 h on ice to allow binding of the virus to the cell surface. The unbound virus was subsequently removed by washing three times with cold PBS. Serial 1:5 dilutions of the indicated ascites fluids of 3E8 and 2D9 were added to the cells and incubated for 1 h on ice. After this incubation, the antibody and detached virus were removed with cold PBS, and RNA extraction and RT-qPCR quantification were performed as described above. As negative control, mAb 3B4 against HAstV1 was used. The arithmetic means and standard deviation error bars from three independent experiments, performed in duplicate (3E8 and 2D9) or as single replicas (3B4) are shown. A Mann-Whitney U test was used to compare samples with mAbs 3E8 or 2D9 to samples with control mAb 3B4.

Expression and purification of chimeric Fabs 3E8, 2D9, and 3B4 Synthetic cDNA encoding the kappa and heavy chain variable regions of each antibody was cloned by Gibson assembly into the pCMV-VRC01 antibody vectors for light and heavy chains, in place of the variable regions of antibody VRC01, a human anti-HIV antibody targeting the gp120 protein ⁴⁹. For the Fab heavy chain, only the variable region followed by the constant heavy 1 region ending with residues “DKKVEPKSC” (SEQ ID NO:82) was included, followed by an AS linker, C-terminal thrombin cleavage site, and a Twin-Strep-tag. Sequences were in-frame with the N-terminal signal sequence. The resulting chimeric Fab expression plasmids, pCMV-Fab_kappa and pCMV-Fab_heavy_VH+CH1, where Fab is 3E8, 2D9, or 3B4, contain the variable regions from the original mouse antibodies and the constant regions from the human IgG1 antibody under the control of the human cytomegalovirus promoter. The plasmids were verified by DNA sequencing. The expression plasmids were used in a ratio of 3:2 kappa chain: heavy VH+CH1 chain to electroporate Chinese Hamster Ovary suspension (CHO-S) cells using the MaxCyte system. Recombinant chimeric Fabs were expressed for 9 days (Fabs 3E8 and 3B4) or 7 days (Fab 2D9) by CHO-S cells growing in CD OptiCHO expression medium supplemented with 1 mM sodium butyrate, 8 mM L-glutamine, 1×HT supplement, and 0.1% Pluronic F68 at 32° C. with 125 rpm shaking. Every 24 h, cells were fed with CHO CD EfficientFeed A supplemented with 7 mM L-glutamine, 5.5% glucose, and 23.4 g/L yeastolate. After 9 days, cells were pelleted and medium containing secreted Fabs was 0.22-μm filtered, buffered to Strep wash buffer (50 mM Tris pH 7.5, 150 mM NaCl, 1 mM EDTA), and supplemented with BioLock (IBA Lifesciences) to mask free biotin in the medium. The samples were 0.22-μm filtered again, each affinity purified on two tandem 5 mL StrepTrap HP columns (GE), and eluted in Strep elution buffer (Strep wash buffer with 2.5 mM desthiobiotin). Fabs 3E8 and 3B4 were dialyzed overnight into 10 mM Tris-HCl pH 7.2, 150 mM NaCl (TBS pH 7.2). Fab 2D9 was dialyzed overnight into 1×PBS pH 7.4 (Sigma-Aldrich).

### GFP-Spike 8 Attachment Inhibition Assay by Fluorescence Microscopy

GFP-Spike 8 was expressed and purified as described previously.⁴⁰ 24-well plates containing fibronectin-treated glass coverslips were seeded with 100,000 Caco-2 cells per well and allowed to adhere overnight. 250 μl samples containing 400 nM GFP-Spike 8 were incubated with three molar excess (1200 nM) of antigen-binding fragments (Fabs) of Spike 8-specific monoclonal antibodies 3E8 and 2D9 and the Fab of Spike 1-specific monoclonal antibody 3B4 for 1 h at room temperature in Dulbecco's phosphate-buffered saline (PBS) (Gibco). PBS alone and 400 nM GFP samples were used as controls for autofluorescence and non-specific binding respectively. Media was aspirated from the cell monolayer and Spike-Fab mixtures were then added to Caco-2 cells and incubated at 4° C. for 1 h. Protein mixtures were removed, and cells were washed with PBS and fixed with 2% paraformaldehyde (ThermoFisher) in PBS for 15 min. Cells were washed with PBS and then stained with Hoechst 33342 dye (ThermoFisher) in DPBS for 30 minutes. Coverslips were washed with PBS, dried, and mounted in Vectashield mounting media on glass microscope slides.

Z-stack images were acquired by using identical acquisition parameters with a Zeiss Axio Imager equipped with an AxioCam 506 monochrome camera using an oil-immersion 100×/1.4 n.a. plan apo objective lens. Z-stack images contained 9 slices at 0.24 μm intervals with GFP and Hoescht channels exposed for 1600 and 95 ms, respectively. GFP signal was collected with a Zeiss Fset38 filter cube and Hoechst signal was collected with a Zeiss Fset49 filter cube. After acquisition, images were deconvolved with AutoQuant X 3D deconvolution software (Media Cybernetics Version X3.1.3) for 10 iterations using the Z montage option. After deconvolution, a median filter with a 2-pixel kernel size was applied to the GFP channel of all images to reduce noise. Linear histogram adjustments were made to the GFP channel using FIJI⁵⁰ such that minimum values were 1300 and maximum values were 7500 to help reduce background fluorescence. Single Z stack slices from representative images were then converted to an RGB image. Images were cropped to identical sizes in FIJI to select representative cells and are representative of data from at least three independent experiments.

Coordinates and structure factors for the scFv 3E8/Spike 8 structure and the scFv 2D9/Spike 8 structure have been deposited in the Protein Data Bank under accession codes 7RK1 and 7RK2, respectively.

### REFERENCES

1. Mendez, E.; Arias, C. F., Astroviruses. In Fields Virology, 2 ed.; Howley, P. M., Ed. Lippincott Williams & Wilkins: 2007; pp 982-1000.
2. Koci, M. D.; Schultz-Cherry, S., Avian astroviruses. Avian Pathol 2002, 31 (3), 213-27.
3. De Benedictis, P.; Schultz-Cherry, S.; Burnham, A.; Cattoli, G., Astrovirus infections in humans and animals—molecular biology, genetic diversity, and interspecies transmissions. Infect Genet Evol 2011, 11 (7), 1529-44.
4. King, A. M. Q.; Lefkowitz, E.; Adams, M. J.; Carstens, E. B., Virus taxonomy: classification and nomenclature of viruses. In Ninth report of the International Committee on Taxonomy of Viruses, 9th edition ed.; Elsevier Inc: Philadelphia, PA, 2011.
5. Koopmans, M. P.; Bijen, M. H.; Monroe, S. S.; Vinje, J., Age-stratified seroprevalence of neutralizing antibodies to astrovirus types 1 to 7 in humans in The Netherlands. Clin Diagn Lab Immunol 1998, 5 (1), 33-7.
6. Kurtz, J. B.; Lee, T. W., Human astrovirus serotypes. Lancet 1984, 2 (8416), 1405.
7. Meyer, L.; Delgado-Cunningham, K.; Lorig-Roach, N.; Ford, J.; DuBois, R. M., Human Astrovirus 1-8 Seroprevalence Evaluation in a United States Adult Population. Viruses 2021, 13 (6).
8. Cortez, V.; Meliopoulos, V. A.; Karlsson, E. A.; Hargest, V.; Johnson, C.; Schultz-Cherry, S., Astrovirus Biology and Pathogenesis. Annu Rev Virol 2017, 4 (1), 327-348.
9. Walter, J. E.; Mitchell, D. K., Astrovirus infection in children. Curr Opin Infect Dis 2003, 16 (3), 247-53.
10. Goodgame, R. W., Viral causes of diarrhea. Gastroenterol Clin North Am 2001, 30 (3), 779-95.
11. Gray, J. J.; Wreghitt, T. G.; Cubitt, W. D.; Elliot, P. R., An outbreak of gastroenteritis in a home for the elderly associated with astrovirus type 1 and human calicivirus. J Med Virol 1987, 23 (4), 377-81.
12. Lewis, D. C.; Lightfoot, N. F.; Cubitt, W. D.; Wilson, S. A., Outbreaks of astrovirus type 1 and rotavirus gastroenteritis in a geriatric in-patient population. J Hosp Infect 1989, 14 (1), 9-14.
13. Marshall, J. A.; Bruggink, L. D.; Sturge, K.; Subasinghe, N.; Tan, A.; Hogg, G. G., Molecular features of astrovirus associated with a gastroenteritis outbreak in an aged-care centre. Eur J Clin Microbiol Infect Dis 2007, 26 (1), 67-71.
14. Dennehy, P. H.; Nelson, S. M.; Spangenberger, S.; Noel, J. S.; Monroe, S. S.; Glass, R. I., A prospective case-control study of the role of astrovirus in acute diarrhea among hospitalized young children. J Infect Dis 2001, 184 (1), 10⁻⁵.
15. Gallimore, C. I.; Taylor, C.; Gennery, A. R.; Cant, A. J.; Galloway, A.; Xerry, J.; Adigwe, J.; Gray, J. J., Contamination of the hospital environment with gastroenteric viruses: comparison of two pediatric wards over a winter season. J Clin Microbiol 2008, 46 (9), 3112-5.
16. Olortegui, M. P.; Rouhani, S.; Yori, P. P.; Salas, M. S.; Trigoso, D. R.; Mondal, D.; Bodhidatta, L.; Platts-Mills, J.; Samie, A.; Kabir, F.; Lima, A.; Babji, S.; Shrestha, S. K.; Mason, C. J.; Kalam, A.; Bessong, P.; Ahmed, T.; Mduma, E.; Bhutta, Z. A.; Lima, I.; Ramdass, R.; Moulton, L. H.; Lang, D.; George, A.; Zaidi, A. K. M.; Kang, G.; Houpt, E. R.; Kosek, M. N.; Network, M.-E., Astrovirus Infection and Diarrhea in 8 Countries. Pediatrics 2018, 141 (1).
17. Mead, P. S.; Slutsker, L.; Dietz, V.; McCaig, L. F.; Bresee, J. S.; Shapiro, C.; Griffin, P. M.; Tauxe, R. V., Food-related illness and death in the United States. Emerg Infect Dis 1999, 5 (5), 607-25.
18. Palombo, E. A.; Bishop, R. F., Annual incidence, serotype distribution, and genetic diversity of human astrovirus isolates from hospitalized children in Melbourne, Australia. J Clin Microbiol 1996, 34 (7), 1750-3.
19. Bosch, A.; Pinto, R. M.; Guix, S., Human astroviruses. Clin Microbiol Rev 2014, 27 (4), 1048-74.
20. Cortez, V.; Freiden, P.; Gu, Z.; Adderson, E.; Hayden, R.; Schultz-Cherry, S., Persistent Infections with Diverse Co-Circulating Astroviruses in Pediatric Oncology Patients, Memphis, Tennessee, USA. Emerg Infect Dis 2017, 23 (2), 288-290.
21. van der Doef, H. P.; Bathoorn, E.; van der Linden, M. P.; Wolfs, T. F.; Minderhoud, A. L.; Bierings, M. B.; Wensing, A. M.; Lindemans, C. A., Astrovirus outbreak at a pediatric hematology and hematopoietic stem cell transplant unit despite strict hygiene rules. Bone Marrow Transplant 2016, 51 (5), 747-50.
22. Brown, J. R.; Morfopoulou, S.; Hubb, J.; Emmett, W. A.; Ip, W.; Shah, D.; Brooks, T.; Paine, S. M.; Anderson, G.; Virasami, A.; Tong, C. Y.; Clark, D. A.; Plagnol, V.; Jacques, T. S.; Qasim, W.; Hubank, M.; Breuer, J., Astrovirus VA1/HMO-C: an increasingly recognized neurotropic pathogen in immunocompromised patients. Clin Infect Dis 2015, 60 (6), 881-8.
23. Naccache, S. N.; Peggs, K. S.; Mattes, F. M.; Phadke, R.; Garson, J. A.; Grant, P.; Samayoa, E.; Federman, S.; Miller, S.; Lunn, M. P.; Gant, V.; Chiu, C. Y., Diagnosis of neuroinvasive astrovirus infection in an immunocompromised adult with encephalitis by unbiased next-generation sequencing. Clin Infect Dis 2015, 60 (6), 919-23.
24. Quan, P. L.; Wagner, T. A.; Briese, T.; Torgerson, T. R.; Hornig, M.; Tashmukhamedova, A.; Firth, C.; Palacios, G.; Baisre-De-Leon, A.; Paddock, C. D.; Hutchison, S. K.; Egholm, M.; Zaki, S. R.; Goldman, J. E.; Ochs, H. D.; Lipkin, W. I., Astrovirus encephalitis in boy with X-linked agammaglobulinemia. Emerg Infect Dis 2010, 16 (6), 918-25.
25. Cordey, S.; Vu, D. L.; Schibler, M.; L'Huillier, A. G.; Brito, F.; Docquier, M.; Posfay-Barbe, K. M.; Petty, T. J.; Turin, L.; Zdobnov, E. M.; Kaiser, L., Astrovirus MLB2, a New Gastroenteric Virus Associated with Meningitis and Disseminated Infection. Emerg Infect Dis 2016, 22 (5), 846-53.
26. Kurtz, J.; Lee, T., Astrovirus gastroenteritis age distribution of antibody. Med Microbiol Immunol 1978, 166 (1-4), 227-30.
27. Kurtz, J. B.; Lee, T. W.; Craig, J. W.; Reed, S. E., Astrovirus infection in volunteers. J Med Virol 1979, 3 (3), 221-30.
28. Mitchell, D. K., Astrovirus gastroenteritis. Pediatr Infect Dis J 2002, 21 (11), 1067-9.
29. Bjorkholm, M.; Celsing, F.; Runarsson, G.; Waldenstrom, J., Successful intravenous immunoglobulin therapy for severe and persistent astrovirus gastroenteritis after fludarabine treatment in a patient with Waldenstrom's macroglobulinemia. Int J Hematol 1995, 62 (2), 117-20.
30. Lewis, T. L.; Greenberg, H. B.; Herrmann, J. E.; Smith, L. S.; Matsui, S. M., Analysis of astrovirus serotype 1 RNA, identification of the viral RNA-dependent RNA polymerase motif, and expression of a viral structural protein. J Virol 1994, 68 (1), 77-83.
31. Jiang, B.; Monroe, S. S.; Koonin, E. V.; Stine, S. E.; Glass, R. I., RNA sequence of astrovirus: distinctive genomic organization and a putative retrovirus-like ribosomal frameshifting signal that directs the viral replicase synthesis. Proc Natl Acad Sci USA 1993, 90 (22), 10539-43.
32. Krishna, N. K., Identification of structural domains involved in astrovirus capsid biology. Viral Immunol 2005, 18 (1), 17-26.
33. Dryden, K. A.; Tihova, M.; Nowotny, N.; Matsui, S. M.; Mendez, E.; Yeager, M., Immature and Mature Human Astrovirus: Structure, Conformational Changes, and Similarities to Hepatitis E Virus. J Mol Biol 2012.
34. Mendez, E.; Fernandez-Luna, T.; Lopez, S.; Mendez-Toss, M.; Arias, C. F., Proteolytic processing of a serotype 8 human astrovirus ORF2 polyprotein. Journal of Virology 2002, 76 (16), 7996-8002.
35. Mendez, E.; Salas-Ocampo, E.; Arias, C. F., Caspases mediate processing of the capsid precursor and cell release of human astroviruses. J Virol 2004, 78 (16), 8601-8.
36. Bass, D. M.; Qiu, S., Proteolytic processing of the astrovirus capsid. J Virol 2000, 74 (4), 1810-4.
37. York, R. L.; Yousefi, P. A.; Bogdanoff, W.; Haile, S.; Tripathi, S.; DuBois, R. M., Structural, Mechanistic, and Antigenic Characterization of the Human Astrovirus Capsid. J Virol 2015, 90 (5), 2254-63.
38. Toh, Y.; Harper, J.; Dryden, K. A.; Yeager, M.; Arias, C. F.; Méndez, E.; Tao, Y. J., Crystal Structure of the Human Astrovirus Capsid Protein. J Virol 2016, 90 (20), 9008-17.
39. Dong, J.; Dong, L.; Mendez, E.; Tao, Y., Crystal structure of the human astrovirus capsid spike. Proceedings of the National Academy of Sciences of the United States of America 2011, 108 (31), 12681-6.
40. Bogdanoff, W. A.; Campos, J.; Perez, E. I.; Yin, L.; Alexander, D. L.; DuBois, R. M., Structure of a Human Astrovirus Capsid-Antibody Complex and Mechanistic Insights into Virus Neutralization. J Virol 2017, 91 (2).
41. Espinosa, R.; López, T.; Bogdanoff, W. A.; Espinoza, M. A.; López, S.; DuBois, R. M.; Arias, C. F., Isolation of neutralizing monoclonal antibodies to human astrovirus and characterization of virus variants that escape neutralization. J Virol 2018.
42. Sanchez-Fauquier, A.; Carrascosa, A. L.; Carrascosa, J. L.; Otero, A.; Glass, R. I.; Lopez, J. A.; San Martin, C.; Melero, J. A., Characterization of a human astrovirus serotype 2 structural protein (VP26) that contains an epitope involved in virus neutralization. Virology 1994, 201 (2), 312-20.
43. Bass, D. M.; Upadhyayula, U., Characterization of human serotype 1 astrovirus-neutralizing epitopes. J Virol 1997, 71 (11), 8666-71.
44. Sievers, F.; Wilm, A.; Dineen, D.; Gibson, T. J.; Karplus, K.; Li, W.; Lopez, R.; McWilliam, H.; Remmert, M.; Söding, J.; Thompson, J. D.; Higgins, D. G., Fast, scalable generation of high-quality protein multiple sequence alignments using Clustal Omega. Mol Syst Biol 2011, 7,539.
45. Robert, X.; Gouet, P., Deciphering key features in protein structures with the new ENDscript server. Nucleic Acids Res 2014, 42 (Web Server issue), W320-4.
46. Meyer, L.; L6pez, T.; Espinosa, R.; Arias, C. F.; Vollmers, C.; DuBois, R. M., Asimplified workflow for monoclonal antibody sequencing. PLoS One 2019, 14 (6), e0218717.
47. Aguilar-Hernandez, N.; López, S.; Arias, C. F., Minimal capsid composition of infectious human astrovirus. Virology 2018, 521, 58-61.
48. Bogdanoff, W. A.; Perez, E. I.; López, T.; Arias, C. F.; DuBois, R. M., Structural Basis for Escape of Human Astrovirus from Antibody Neutralization: Broad Implications for Rational Vaccine Design. J Virol 2018, 92 (1).
49. Wu, X.; Yang, Z. Y.; Li, Y.; Hogerkorp, C. M.; Schief, W. R.; Seaman, M. S.; Zhou, T.; Schmidt, S. D.; Wu, L.; Xu, L.; Longo, N. S.; McKee, K.; O'Dell, S.; Louder, M. K.; Wycuff, D. L.; Feng, Y.; Nason, M.; Doria-Rose, N.; Connors, M.; Kwong, P. D.; Roederer, M.; Wyatt, R. T.; Nabel, G. J.; Mascola, J. R., Rational design of envelope identifies broadly neutralizing human monoclonal antibodies to HIV-1. Science 2010, 329 (5993), 856-61.
50. Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y.; White, D. J.; Hartenstein, V.; Eliceiri, K.; Tomancak, P.; Cardona, A., Fiji: an open-source platform for biological-image analysis. Nat Methods 2012, 9 (7), 676-82.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. An antibody that specifically binds to a human astrovirus (HAstV) capsid spike protein and competes for binding to the HAstV capsid spike protein with an antibody comprising:
a variable heavy chain (V_{H}) polypeptide comprising
a V_{H} CDR1 comprising the amino acid sequence GFSLTSYGVH (SEQ ID NO:2),
a V_{H} CDR2 comprising the amino acid sequence VIWSGGSTDYNAAFIS (SEQ ID NO:3), and
a V_{H} CDR3 comprising the amino acid sequence NSLLDAMDY (SEQ ID NO:4), and
a variable light chain (V_{L}) polypeptide comprising
a V_{L} CDR1 comprising the amino acid sequence KASQSVSNAVA (SEQ ID NO:6),
a V_{L} CDR2 comprising the amino acid sequence YASNRYT (SEQ ID NO:7), and
a V_{L} CDR3 comprising the amino acid sequence QQDYSSPLT (SEQ ID NO:8);
a variable heavy chain (V_{H}) polypeptide comprising
a V_{H} CDR1 comprising the amino acid sequence GYTFTSYWMH (SEQ ID NO:10),
a V_{H} CDR2 comprising the amino acid sequence EINPSSGRANYNEKFKN (SEQ ID NO:11), and
a V_{H} CDR3 comprising the amino acid sequence DYYAMDY (SEQ ID NO:12), and
a variable light chain (V_{L}) polypeptide comprising
a V_{L} CDR1 comprising the amino acid sequence RASQSISNNLH (SEQ ID NO:14),
a V_{L} CDR2 comprising the amino acid sequence SASQSIS (SEQ ID NO:15), and
a V_{L} CDR3 comprising the amino acid sequence QQTNSWPLT (SEQ ID NO:16);
a variable heavy chain (V_{H}) polypeptide comprising
a V_{H} CDR1 comprising the amino acid sequence GYSITSGYYWN (SEQ ID NO:18),
a V_{H} CDR2 comprising the amino acid sequence YISYDGSNNYNPSLKN (SEQ ID NO:19), and
a V_{H} CDR3 comprising the amino acid sequence FYDGYDY (SEQ ID NO:20), and
a variable light chain (V_{L}) polypeptide comprising
a V_{L} CDR1 comprising the amino acid sequence KASQDVSTAVA (SEQ ID NO:22),
a V_{L} CDR2 comprising the amino acid sequence WASTRHT (SEQ ID NO:23), and
a V_{L} CDR3 comprising the amino acid sequence QQHYSTPFT (SEQ ID NO:24);
a variable heavy chain (V_{H}) polypeptide comprising
a V_{H} CDR1 comprising the amino acid sequence GFSLTTFGIH (SEQ ID NO:26),
a V_{H} CDR2 comprising the amino acid sequence VIWAAGTTNYNSTLKS (SEQ ID NO:27), and
a V_{H} CDR3 comprising the amino acid sequence EDYDYFFGLDY (SEQ ID NO:28), and
a variable light chain (V_{L}) polypeptide comprising
a V_{L} CDR1 comprising the amino acid sequence RSSTGAVTTSNYAS (SEQ ID NO:30),
a V_{L} CDR2 comprising the amino acid sequence GTNNRAP (SEQ ID NO:31), and
a V_{L} CDR3 comprising the amino acid sequence ALWFSNHWV (SEQ ID NO:32);
a variable heavy chain (V_{H}) polypeptide comprising
a V_{H} CDR1 comprising the amino acid sequence GFSLTSYGVH (SEQ ID NO:1),
a V_{H} CDR2 comprising the amino acid sequence VIWADGSTNYNSALMS (SEQ ID NO:34), and
a V_{H} CDR3 comprising the amino acid sequence WTYGDYFDY (SEQ ID NO:35), and
a variable light chain (V_{L}) polypeptide comprising
a V_{L} CDR1 comprising the amino acid sequence SASQGISNYLN (SEQ ID NO:37),
a V_{L} CDR2 comprising the amino acid sequence YTSSLHS (SEQ ID NO:38), and
a V_{L} CDR3 comprising the amino acid sequence QQYSKLPYT (SEQ ID NO:39);
a variable heavy chain (V_{H}) polypeptide comprising
a V_{H} CDR1 comprising the amino acid sequence GFTFSYFGMH (SEQ ID NO:41),
a V_{H} CDR2 comprising the amino acid sequence YISSGSNTIYYADTVKG (SEQ ID NO:42), and
a V_{H} CDR3 comprising the amino acid sequence AYYGNHYYAMDF (SEQ ID NO:43), and
a variable light chain (V_{L}) polypeptide comprising
a V_{L} CDR1 comprising the amino acid sequence RASENIYSNLA (SEQ ID NO:45),
a V_{L} CDR2 comprising the amino acid sequence AARDLAT (SEQ ID NO:46), and
a V_{L} CDR3 comprising the amino acid sequence QHFWETPWT (SEQ ID NO:47); or
a variable heavy chain (V_{H}) polypeptide comprising
a V_{H} CDR1 comprising the amino acid sequence GFTFSDYYMY (SEQ ID NO:49),
a V_{H} CDR2 comprising the amino acid sequence TISDGGFSTYYPDSVKG (SEQ ID NO:50), and
a V_{H} CDR3 comprising the amino acid sequence FGAYSTF (SEQ ID NO:51), and
a variable light chain (V_{L}) polypeptide comprising
a V_{L} CDR1 comprising the amino acid sequence RASSSVSYMY (SEQ ID NO:53),
a V_{L} CDR2 comprising the amino acid sequence ATSNLAS (SEQ ID NO:54), and
a V_{L} CDR3 comprising the amino acid sequence QQWSSDPPMT (SEQ ID NO:55).

2. The antibody of claim 1, wherein the antibody comprises:
a variable heavy chain (V_{H}) polypeptide comprising
a V_{H} CDR1 comprising the amino acid sequence GFSLTSYGVH (SEQ ID NO:2),
a V_{H} CDR2 comprising the amino acid sequence VIWSGGSTDYNAAFIS (SEQ ID NO:3), and
a V_{H} CDR3 comprising the amino acid sequence NSLLDAMDY (SEQ ID NO:4), and
a variable light chain (V_{L}) polypeptide comprising
a V_{L} CDR1 comprising the amino acid sequence KASQSVSNAVA (SEQ ID NO:6),
a V_{L} CDR2 comprising the amino acid sequence YASNRYT (SEQ ID NO:7), and
a V_{L} CDR3 comprising the amino acid sequence QQDYSSPLT (SEQ ID NO:8);
a variable heavy chain (V_{H}) polypeptide comprising
a V_{H} CDR1 comprising the amino acid sequence GYTFTSYWMH (SEQ ID NO:10),
a V_{H} CDR2 comprising the amino acid sequence EINPSSGRANYNEKFKN (SEQ ID NO:11), and
a V_{H} CDR3 comprising the amino acid sequence DYYAMDY (SEQ ID NO:12), and
a variable light chain (V_{L}) polypeptide comprising
a V_{L} CDR1 comprising the amino acid sequence RASQSISNNLH (SEQ ID NO:14),
a V_{L} CDR2 comprising the amino acid sequence SASQSIS (SEQ ID NO:15), and
a V_{L} CDR3 comprising the amino acid sequence QQTNSWPLT (SEQ ID NO:16);
a variable heavy chain (V_{H}) polypeptide comprising
a V_{H} CDR1 comprising the amino acid sequence GYSITSGYYWN (SEQ ID NO:18),
a V_{H} CDR2 comprising the amino acid sequence YISYDGSNNYNPSLKN (SEQ ID NO:19), and
a V_{H} CDR3 comprising the amino acid sequence FYDGYDY (SEQ ID NO:20), and
a variable light chain (V_{L}) polypeptide comprising
a V_{L} CDR1 comprising the amino acid sequence KASQDVSTAVA (SEQ ID NO:22),
a V_{L} CDR2 comprising the amino acid sequence WASTRHT (SEQ ID NO:23), and
a V_{L} CDR3 comprising the amino acid sequence QQHYSTPFT (SEQ ID NO:24);
a variable heavy chain (V_{H}) polypeptide comprising
a V_{H} CDR1 comprising the amino acid sequence GFSLTTFGIH (SEQ ID NO:26),
a V_{H} CDR2 comprising the amino acid sequence VIWAAGTTNYNSTLKS (SEQ ID NO:27), and
a V_{H} CDR3 comprising the amino acid sequence EDYDYFFGLDY (SEQ ID NO:28), and
a variable light chain (V_{L}) polypeptide comprising
a V_{L} CDR1 comprising the amino acid sequence RSSTGAVTTSNYAS (SEQ ID NO:30),
a V_{L} CDR2 comprising the amino acid sequence GTNNRAP (SEQ ID NO:31), and
a V_{L} CDR3 comprising the amino acid sequence ALWFSNHWV (SEQ ID NO:32);
a variable heavy chain (V_{H}) polypeptide comprising
a V_{H} CDR1 comprising the amino acid sequence GFSLTSYGVH (SEQ ID NO:1),
a V_{H} CDR2 comprising the amino acid sequence VIWADGSTNYNSALMS (SEQ ID NO:34), and
a V_{H} CDR3 comprising the amino acid sequence WTYGDYFDY (SEQ ID NO:35), and
a variable light chain (V_{L}) polypeptide comprising
a V_{L} CDR1 comprising the amino acid sequence SASQGISNYLN (SEQ ID NO:37),
a V_{L} CDR2 comprising the amino acid sequence YTSSLHS (SEQ ID NO:38), and
a V_{L} CDR3 comprising the amino acid sequence QQYSKLPYT (SEQ ID NO:39);
a variable heavy chain (V_{H}) polypeptide comprising
a V_{H} CDR1 comprising the amino acid sequence GFTFSYFGMH (SEQ ID NO:41),
a V_{H} CDR2 comprising the amino acid sequence YISSGSNTIYYADTVKG (SEQ ID NO:42), and
a V_{H} CDR3 comprising the amino acid sequence AYYGNHYYAMDF (SEQ ID NO:43), and
a variable light chain (V_{L}) polypeptide comprising
a V_{L} CDR1 comprising the amino acid sequence RASENIYSNLA (SEQ ID NO:45),
a V_{L} CDR2 comprising the amino acid sequence AARDLAT (SEQ ID NO:46), and
a V_{L} CDR3 comprising the amino acid sequence QHFWETPWT (SEQ ID NO:47); or
a variable heavy chain (V_{H}) polypeptide comprising
a V_{H} CDR1 comprising the amino acid sequence GFTFSDYYMY (SEQ ID NO:49),
a V_{H} CDR2 comprising the amino acid sequence TISDGGFSTYYPDSVKG (SEQ ID NO:50), and
a V_{H} CDR3 comprising the amino acid sequence FGAYSTF (SEQ ID NO:51), and
a variable light chain (V_{L}) polypeptide comprising
a V_{L} CDR1 comprising the amino acid sequence RASSSVSYMY (SEQ ID NO:53),
a V_{L} CDR2 comprising the amino acid sequence ATSNLAS (SEQ ID NO:54), and
a V_{L} CDR3 comprising the amino acid sequence QQWSSDPPMT (SEQ ID NO:55).

3. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:1; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:5.

4. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:9; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:13.

5. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:21.

6. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:25; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:29.

7. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:33; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:36.

8. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:40; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:44.

9. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:48; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:52.

10. The antibody of any one of claims 1 to 9, wherein the antibody is a monoclonal antibody.

11. The antibody of any one of claims 1 to 10, wherein the antibody is a humanized antibody.

12. The antibody of any one of claims 1 to 11, wherein the antibody is an IgG.

13. The antibody of claim 12, wherein the antibody comprises a human Fc domain.

14. The antibody of claim 13, wherein the human Fc domain is a human IgG1 Fc domain.

15. The antibody of any one of claims 1 to 11, wherein the antibody is selected from the group consisting of: a Fab, a F(ab′)₂, and a F(ab′).

16. The antibody of any one of claims 1 to 11, wherein the antibody is a single chain antibody.

17. The antibody of claim 16, wherein the single chain antibody is an scFv.

18. The antibody of any one of claims 1 to 17, wherein the antibody is a bispecific antibody comprising a first antigen-binding domain comprising a V_{H} polypeptide-V_{L} polypeptide pair as defined in any one of claims 1 to 9.

19. The antibody of claim 18, wherein the bispecific antibody comprises a second antigen-binding domain that specifically binds a different epitope on the HAstV capsid spike protein as compared to the epitope specifically bound by the first antigen-binding domain.

20. The antibody of claim 18, wherein the bispecific antibody comprises a second antigen-binding domain that specifically binds an antigen other than a HAstV capsid spike protein.

21. A fusion protein, comprising:
a chain of an antibody of any one of claims 1 to 20 fused to a heterologous sequence of amino acids.

22. The fusion protein of claim 21, wherein the heterologous sequence of amino acids is fused to the C-terminus of the chain of the antibody.

23. The fusion protein of claim 21 or claim 22, wherein the antibody is the single chain antibody of claim 16 or 17.

24. A conjugate, comprising:
an antibody of any one of claims 1 to 20 or a fusion protein of any one of claims 21 to 23; and
an agent conjugated to the antibody or fusion protein.

25. The conjugate of claim 24, wherein the agent is a detectable label or a half-life extending moiety.

26. The conjugate of claim 24 or claim 25, wherein the agent is conjugated to the antibody via a non-cleavable linker.

27. The conjugate of claim 24 or claim 25, wherein the agent is conjugated to the antibody via a cleavable linker.

28. A nucleic acid encoding a variable heavy chain (V_{H}) polypeptide, a variable light chain (V_{L}) polypeptide, or both, of an antibody of any one of claims 1 to 20.

29. A nucleic acid encoding the fusion protein of any one of claims 21 to 23.

30. An expression vector comprising the nucleic acid of claim 28 or claim 29.

31. A cell comprising:
the nucleic acid of claim 28 or claim 29; or
the expression vector of claim 30.

32. A cell comprising:
a first nucleic acid encoding the variable heavy chain (V_{H}) polypeptide of an antibody of any one of claims 1 to 15; and
a second nucleic acid encoding the variable light chain (V_{L}) polypeptide of the antibody.

33. The cell of claim 32, comprising:
a first expression vector comprising the first nucleic acid; and
a second expression vector comprising the second nucleic acid.

34. A method of producing the antibody of any one of claims 1 to 15, comprising culturing the cell of any one of claims 31 to 33 under conditions suitable for the cell to express the antibody, wherein the antibody is produced.

35. A pharmaceutical composition, comprising:
the antibody of any one of claims 1 to 20; and
a pharmaceutically acceptable carrier.

36. A pharmaceutical composition, comprising:
the fusion protein of any one of claims 21 to 23; and
a pharmaceutically acceptable carrier.

37. A pharmaceutical composition, comprising:
the conjugate of any one of claims 24 to 27; and
a pharmaceutically acceptable carrier.

38. A kit, comprising:
the pharmaceutical composition of any one of claims 35 to 37; and
instructions for administering the pharmaceutical composition to an individual in need thereof.

39. The kit of claim 38, wherein the pharmaceutical composition is present in one or more unit dosages.

40. The kit of claim 38, wherein the pharmaceutical composition is present in two or more unit dosages.

41. A method, comprising:
administering to an individual having a human astrovirus (HAstV) infection the antibody of any one of claims 1 to 20, the fusion protein of any one of claims 21 to 23, or the conjugate of any one of claims 24 to 27.

42. A method of treating an individual having a human astrovirus (HAstV) infection, the method comprising:
administering to the individual a therapeutically effective amount of the antibody of any one of claims 1 to 20, the fusion protein of any one of claims 21 to 23, or the conjugate of any one of claims 24 to 27.

43. The method according to claim 41 or claim 42, wherein the infection is a HAstV serotype 1 (HAstV1) infection, a HAstV serotype 2 (HAstV2) infection, a HAstV serotype 3 (HAstV3) infection, a HAstV serotype 4 (HAstV4) infection, a HAstV serotype 5 (HAstV5) infection, a HAstV serotype 6 (HAstV6) infection, a HAstV serotype 7 (HAstV7) infection, or a HAstV serotype 1 (HAstV8) infection.

44. The method according to claim 41 or claim 42, wherein the infection is a HAstV serotype 1 (HAstV1) infection.

45. The method according to any one of claims 41 to 44, wherein the individual has astroviral gastroenteritis.

46. The method according to any one of claims 41 to 45, wherein the individual has astroviral encephalitis.
